(19) United States
(12) Patent Application Publication
(10) Pub. No.: US 2011/0046101 A1
(43) Pub. Date: Feb. 24, 2011

(54) BATE-LACTAMASE INHIBITORS

(76) Inventors: Gary I. Dmitrienko, Waterloo (CA); Jarrod W. Johnson, Port Colborne (CA); Timothy R. Ramadhar, Waterloo (CA); Thammaiah Viswanatha, Waterloo (CA); Sundaramma Viswanatha, Waterloo (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/441,622

(22) PCT Filed: Mar. 17, 2008
(86) PCT No.: PCT/CA2008/000515

§ 371 Date: Apr. 6, 2010

(51) Int. Cl. A61K 31/166; A61K 31/36; A61K 31/455; A61K 31/43; A61K 31/545; A61K 31/5365; A61K 31/407; A61K 31/431; A61P 31/04; C12N 9/99

(52) U.S. Cl. 514/196; 514/615; 514/466; 514/355; 514/192; 514/200; 514/210.08; 514/210.09; 435/184

(57) ABSTRACT

The present invention relates to broad spectrum β-lactamase inhibitors. More particularly, the invention relates to inhibitors of Class B metallo (MBL) and Class D (OXA) β-lactamases. A method of treating a bacterial infection is provided, wherein the method comprises administering to a mammalian patient in need of such treatment a compound of formula (I)

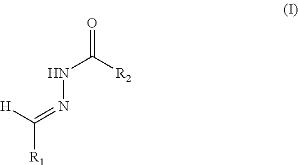

wherein
R₁ is selected from

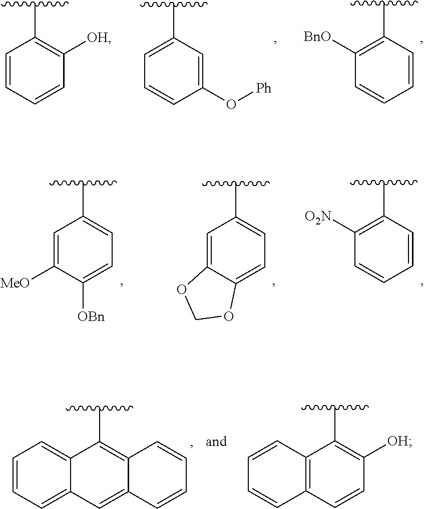

R₂ is selected from

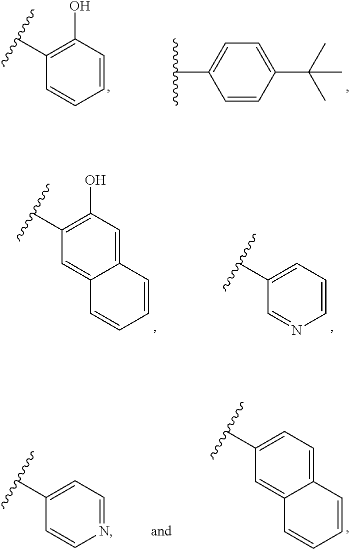

with certain provisos as herein defined;
in combination with a pharmaceutically acceptable β-lactam antibiotic in an amount which is effective for treating the bacterial infection.

### CROSS-REFERENCE TO RELATED APPLICATIONS

This application is the U.S. National Stage of International Application No. PCT/CA2008/000515, filed Mar. 17, 2008 and also claims priority from U.S. Provisional Application No. 61/064,620, filed Mar. 17, 2008. The entire disclosures of each the aforesaid applications are incorporated by reference in the present application.

### FIELD OF THE INVENTION

The present invention relates to broad spectrum β-lactamase inhibitors. More particularly, the invention relates to inhibitors of Class B metallo (MBL) and Class D (OXA) β-lactamases.

### BACKGROUND OF THE INVENTION

The β-lactam antibiotics constitute one of the three largest classes of clinically useful antibiotics along with the fluoroquinolones and macrolides. It is estimated that >50% of all antibiotic prescriptions are for β-lactams. Since the discovery of the naturally occurring penicillins such as penicillin G, a number of significant structural variants, each retaining the essential β-lactam ring, have been discovered and have found specific niches in chemotherapeutic applications (FIG. 1). Dalhoff, A. et al. provide a recent overview of the development of the major classes of β-lactam antibiotics from a medicinal chemistry perspective (“The art of fusion: from penams and cephems to penems.” Chemotherapy 2003, 49, 105).

Since their introduction into standard clinical practice, shortly after the second world war, these antibiotics, which combine the remarkable properties of oral bioavailability (in most cases), high antibiotic potency and relatively low toxicity to the host, have had an enormous impact on the maintenance of human health. As a result, the prospect that bacteria can develop or acquire high levels of resistance to these and other antibiotics is indeed disquieting. For reviews of resistance to β-lactam antibiotics see: (a) Bush, K. 2008 “Extended-spectrum β-lactamases in North America, 1987-2006” Clin. Microbiol. Infect. (14) (Suppl. 1): 134-143 (b) Fisher, J. F., Meroueh, S. O., Mobashery, S. 2005 “Bacterial Resistance to β-Lactam Antibiotics: Compelling Opportunism, Compelling Opportunity” Chem. Rev., 105, 395-424 and references to earlier reviews therein; (c) Poole, K. 2004, “Resistance to β-lactam antibiotics.” Cell Mol. Life. Sci. 61(17):2200-23; (d) Hancock, R. 1997 “The bacterial outer membrane as a drug barrier.” Trends Microbiol. 5, 37-42. A brief but very interesting history of the discovery of the major classes of clinically useful antibiotics and the emergence of resistance to them is presented by Walsh and Wright in the preface to the February 2005 issue of Chemical Reviews which is devoted entirely to reviews of antibiotic resistance mechanisms (Walsh, C. T.; Wright, G. D. 2005 “Introduction: Antibiotic Resistance” Chem. Rev. (Editorial) 105, 391-394).

Various studies have revealed that antibiotic resistance arises typically by three mechanisms: 1) active trans-membrane efflux of the drug; 2) reduction in sensitivity to the drug by modification of the antibiotic target through mutation; and 3) expression of enzymes capable of destruction of the antibiotic ((a) Fisher, J. F., Meroueh, S. O., Mobashery, S. 2005 “Bacterial Resistance to β-Lactam Antibiotics: Compelling Opportunism, Compelling Opportunity.” Chem. Rev., 105, 395-424 and references to earlier reviews therein; (b) Poole, K. 2004, “Resistance to β-lactam antibiotics.” Cell Mol Life Sci. 61, 2200-23; (c) Hancock, R. 1997 “The bacterial outer membrane as a drug barrier.” Trends Microbiol. 5, 37-42). In the case of the β-lactam antibiotics, it has been shown that all three mechanisms play a role to varying degrees. It is generally agreed that the third mechanism, mediated in this case by a variety of hydrolytic enzymes collectively referred to as the β-lactamases, is the single most important cause of high level bacterial resistance to β-lactams.

The ability of some bacteria to effect inactivation of β-lactam antibiotics, through hydrolysis of the β-lactam ring system in penicillins to yield the corresponding penicilloic acid (Scheme 1), was noted very early on in the history of the study of these microbial natural products (Abraham, E. P., Chain, E. B. “An enzyme from bacteria able to destroy penicillin.” Nature 1940, 146, 837).

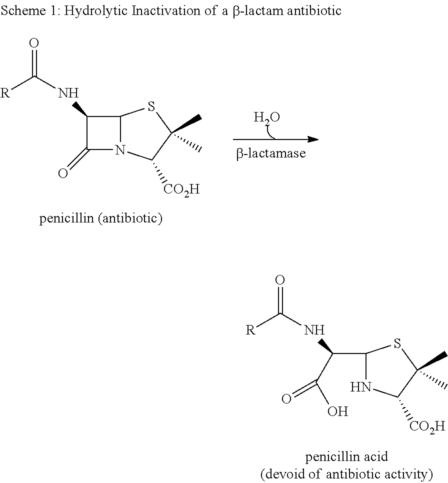

Since those very early indications of the existence of such a potential resistance mechanism, widespread use and abuse of these antibiotics has led to the emergence of a large number of bacterial strains exhibiting high levels of resistance to β-lactams as a consequence of harbouring a β-lactamase gene. It has been estimated that the number of known β-lactamases is approaching 500 (Spencer, J. and Walsh, T. R. 2006 “A New Approach to the Inhibition of Metallo-β-lactamases” Angew. Chem. Int. Ed 45, 1022-1026). The recognition that some β-lactamase genes are plasmid encoded raised concerns in the early 1980's that horizontal transfer of the antibiotic resistance genes would lead to proliferation off β-lactam antibiotic resistant organisms. This has indeed proven to be the case, and from the mid-1980s to 2000 the number of different plasmid-mediated β-lactamases detected in clinical isolates rose from 19 to 255 (Payne, D. J., Du, W., Bateson, J. H. β-Lactamase epidemiology and the utility of established and novel β-lactamase inhibitors.” Exp. Opin. Invest. Drugs 2000, 9, 247-61).

The β-lactamases have been classified by Ambler into four groups: A, B, C and D. The A, C and D classes are all enzymes that employ an active site serine residue as a nucleophile in their catalytic mechanism, in a process somewhat akin to the well-known chymotrypsin ‘acyl enzyme” mechanism. The Class B enzymes employ an active site zinc ion in their catalytic apparatus. The β-lactamases which were first recognized as therapeutic problems were largely of the A type, so initial efforts at combating β-lactam antibiotic resistance were focused on the serine enzymes.

A number of lines of investigation led to the discovery of several so-called mechanism-based inhibitors for the serine β-lactamases, such as sulbactam, tazobactam and clavulanic acid (FIG. 2). These, used in combination with existing penicillins, have served remarkably well to allay the concerns about β-lactamase resistance for the past 25 years, since their introduction into clinical use. For the most part, the Class A β-lactamases have remained susceptible to these inhibitors, although a number of reports of inhibitor-resistant Class A (IRT)-type producing organisms have appeared (Arpin, C., Labia, R., Dubois, V., Noury, P., Souquet, M., Quentin, C. 2002 “TEM-80, a novel inhibitor-resistant β-lactamase in a clinical isolate of Enterobacter cloacae” Antimicrob. Agents Chemother. 46, 1183-9).

In parallel with the development of β-lactamase inhibitors, extensive efforts in various pharmaceutical laboratories to modify the β-lactam systems in order to create antibiotics with broader antibiotic spectrum and lower susceptibility to the β-lactamases were carried out with significant success. Of particular interest was the development of the carbapenems (e.g. imipenem and meropenem, FIG. 1).

The carbapenem ring system was first identified in the structure of the novel, naturally occurring β-lactam antibiotic thienamycin, discovered by scientists at Merck in the U.S. (Kahan, J. S., Kahan, F. M., Stapley, E. O., Goegelman, R. T., Hernandez, S. U.S. Pat. No. 3,950,357, 1976; Chem. Abstr. 1976, 85, 92190t). An inherent chemical instability in thienamycin was eventually attributed to the primary amino group. This problem was solved by conversion of the amino group into the less nucleophilic formamidine group to give imipenem. Imipenem was found to be degraded in vivo by an enzyme called renal dehydropeptidase-I (DHP-I), necessitating the inclusion of a DHP-I inhibitor, cilastatin, in clinical application of imipenem. Later the Astra-Zeneca group discovered that introduction of a beta-oriented methyl group into the five-membered ring of the carbapenem system led to a substantial reduction in susceptibility to hydrolysis by DHP-I, so that such compounds could be administered without the need for a DHP-I inhibitor. This led to the introduction of meropenem into the antibiotic market. Imipenem/cilastatin and meropenem have been found to exhibit an exceptionally broad spectrum of antibiotic potency against pathogenic bacteria, with meropenem exhibiting superior activity against P. aeruginosa and effectiveness in CNS infections where imipenem/cilastatin was contraindicated. Very important also was the observation that the antibiotic effectiveness of these carbapenems extended to organisms that were resistant to other β-lactam antibiotics as a result of production of serine β-lactamases. Thus the carbapenems have emerged as “drugs of last resort” in treatment of serious infections by antibiotic resistant organisms (Edwards, J. R., Betts, M. J. 2000 “The carbapenems: the pinnacle of β-lactam antibiotics or room for improvement?” J. Antimcrob. Chemother. 45, 1-4).

The common occurrence of β-lactamase producing organisms in hospital settings has led to the significant use of the carbapenems to treat serious hospital-acquired, known as nosocomial, infections. Among the serious nosocomial infections are those caused by opportunistic bacteria which are normally harmless towards healthy individuals but which cause serious, potentially fatal, infection in patients with diminished immune systems, including burn victims, AIDS patients, cancer patients, transplant patients and those with lung diseases such as cystic fibrosis.

The emergence of bacteria capable of producing β-lactamases with potent carbapenemase activity has created a great deal of concern about the possibility of the development of high level resistance to these drugs of last resort, leaving the antibiotic cupboard essentially bare in the event of serious nosocomial infections (Queenan A. M., Bush K. 2007 “Carbapenemases: the Versatile β-lactamases” Clin. Microbiol. Rev. 28, 440-458; Jones R. N., Biedenbach D. J., Sader H. S., Fritsche T. R., Toleman M. A., Walsh T. R. 2005 “Emerging epidemic of metallo-β-lactamase-mediated resistances” Diagn Microbiol Infect Dis. 51, 77-84; Livermore, D. M., Woodford, N. 2000 “Carbapenemases: a problem in waiting?” Curr. Opin. Microbiol. 5, 489-95; Livermore, D. M. 2002 “Multiple mechanisms of antimicrobial resistance in Pseudomonas aeruginosa: our worst nightmare?” Clin. Infect. Dis. 34, 634-40; Nordmann P., Poirel L. 2002 “Emerging carbapenemases in Gram-negative aerobes.” Clin. Microbiol. Infect. 8, 321-31).

Although the Class B metallo-β-lactamases have emerged as the most widely feared (Jones, R. N., Biedenbach, D. J., Sader, H. S., Fritsche, T. R., Toleman, M. A., Walsh, T. R. 2005 “Emerging epidemic of metallo-β-lactamase-mediated resistance” Diag. Microbiol. Infect. Dis. 51, 77-84), there is substantial concern about broad spectrum serine β-lactamases of the D-class. The Class-D enzymes are now referred to as oxacillinases (OXAs) in recognition of their ability to hydrolyze oxacillin and cloxacillin (see FIG. 1 for structures) two to four times faster than classical penicillins which possess smaller and less hydrophobic side chains (Sun, T., Nukuga, M., Mayama, K., Braswell, E. H., Knox, J. R. 2003 “Comparison of β-lactamases Classes A and D: 1.5 Å crystallographic structure of the class D OXA-1 oxacillinase” Protein Sci., 12, 82-91). The concern about the OXAs arises for several reasons: 1) numerous members of this growing class (there are now more than 125 variants amongst clinical isolates) of serine-type β-lactamases are relatively resistant to the commercial mechanism-based β-lactamase inhibitors which were originally designed for Class A serine β-lactamases; 2) numerous members of this class exhibit a broad spectrum of activity against most of the commercial β-lactam antibiotics including the carbapenems; 3) increasing numbers of the Class-D enzymes are being found in the clinic, primarily located on plasmids or integrons, suggesting a strong potential for wide dispersal by horizontal gene transfer (Paetzel, M., Danel, F., de Castro, F., Mosimann, S. C., Page, M. G. P., Strynadka, N. C. J. 2000 “Crystal structure of the class D β-lactamase OXA-10” Nature Struct. Biol. 7, 918-925).

Extensive structured screening of antibiotic resistant infections from hospitals world-wide (e.g. the SENTRY and MYSTIC programs) has led to the identification and characterization of an ever increasing variety of MBLs. The SENTRY program monitors the emergence of antibiotic resistant strains of bacteria in hospitals in North and South America, Europe, Australia, Africa, and Asia with a particular focus on identifying carbapenemases, including MBLs and Class D (OXA-type) enzymes. The MYSTIC program, sponsored by Astra-Zeneca, involves 52 hospitals in 19 countries and tracks resistance development to the clinically useful carbapenem, meropenem. (Turner P J, Greenhalgh J. M., Edwards J. R., McKellar J. 1999 “The MYSTIC (Meropenem Yearly Susceptibility Test Information Collection) program” Int. J. Antimicrob. Agents (13):117-25)

Four classes of MBLs identified as being especially troubling are the IMP (Tysall, L., Stockdale M. W., Chadwick P. R., Palepou, M. F., Towner, K. J., Livermore, D. M., Woodford, N. 2002 “IMP-1 carbapenemase detected in an Acinetobacter clinical isolate from the UK” J. Antimicrob. Chemother. 49:217-8), VIM (Docquier, J. D., Lamotte-Brasseur, J., Galleni, M., Amicosante, G., Frere, J. M., Rossolini, G. M. 2003 “On functional and structural heterogeneity of VIM-type metallo-β-lactamases.” J. Antimicrob. Chemother. 51:257-66), SPM-1 (Murphy, T. A., Simm, A. M., Toleman, M. A., Jones, R. N., Walsh, T. R. 2003 “Biochemical characterization of the acquired metallo-tβ-lactamase SPM-1 from Pseudomonas aeruginosa” Antimicrob. Agents Chemother. 47, 582-7) and, more recently, GIM-1 (Castanheira, M., Toleman, M. A., Jones, R. N., Schmidt, F. J., Walsh, T. R. 2004 “Molecular characterization of a beta-lactamase gene, blaGIM-1, encoding a new subclass of metallo-β-lactamase” Antimicrob. Agents Chemother. 48, 4654-61) types. These MBLs are genetically mobile with the MBL genes being found as part of gene cassettes in class 1 or 3 integrons (Fluit, A. C., Schmitz, F.-J. 2004 “Resistance integrons and super-integrons” Clin. Microbiol. Infect.; 10: 272-288). The SENTRY program has yielded a recent overview of the spread of MBL-mediated resistance in what has been termed an “emerging epidemic” (Jones, R. N., Biedenabach, D. J., Sader, H. S., Fritsche, T. R., Toleman, M. A., Walsh, T. R. 2005 “Emerging epidemic of metallo-β-lactamase-mediated resistance” Diag. Microbiol. Infect. Dis. 51, 77-84). For example, resistance arising form the IMP-type MBL was at one time restricted to Japan, but has now been found in Argentina, Brazil, Italy, Taiwan, China, Hong Kong, Singapore, Portugal, and Canada. Whereas IMP-1 was first detected in a Seratia marscenscens strain, IMP-like MBLs are now found also in various strains of Pseudomonas, Acinetobacter, Klebsiela, Citrobacter, Achromobacter and Shigella. The VIM-type MBLs are now detected in drug resistant clinical isolates from France, Italy, Greece, Spain, Korea, Taiwan, Poland, Venezuela, Chile, and the United States. The more recently discovered SPM-1 MBL is localized to hospitals in Brazil, and GIM-1, the most recent member of the genetically mobile MBLs, is being reported only in Germany thus far (Kahan, J. S., Kahan, F. M., Stapley, E. O., Goegelman, R. T., Hernandez, S. U.S. Pat. No. 3,950,357, 1976; Chem. Abstr. 1976, 85, 92190t; Nordmann, P., Poirel, L. 2002 “Emerging carbapenemases in Gram-negative aerobes” Clin. Microbiol. Infect. 8, 321-331).

Recently, as part of the CANCER antimicrobial surveillance program in North America, Walsh and co-workers at the Department of Pathology and Microbiology at the University of Bristol isolated P. aeruginosa strain 07-406 from a clinical isolate from a 58-year-old woman suffering from cancer in Texas. This bacterial strain was found to be resistant to all antibiotics except for polymixin B and to harbour not only the first known MBL in North America, VIM-7 (Toleman, M. A., Rolston, K., Jones, R. N., Walsh, T. R. 2004 “Characterization of blaVIM-7 from Pseudomonas aeruginosa isolated in the United States An evolutionarily distinct metallo-[β-lactamase gene”. Antimicrob. Agents Chemother. 48, 329-332), but also a new variant of the OXA-type of β-lactamase, OXA-45 (Toleman, M. A., Rolston, K., Jones, R. N., Walsh, T. R. 2003 “Molecular and Biochemical Characterization of OXA-45, an Extended-Spectrum Class 2d’ β-Lactamase in Pseudomonas aeruginosa” Antimicrob. Agents Chemother., 47, 2859-2863). It was pointed out that the combination of blaOXA-45 and the VIM-7 MBL gene on a small broad-host-range multicopy plasmid gives P. aeruginosa 07-406 resistance against all β-lactam antibiotics and makes this plasmid a very attractive acquisition for other bacteria trying to survive against the hostile environment of modern anti-infective therapy.

Progress in the inhibition of MBLs has been recently reviewed by Toney (Toney, J. H., Moloughney, J. G. 2004 “Metallo-β-lactamase inhibitors: promise for the future?” Curr. Opin. Investig. Drugs, 8, 823-6). Much of the work reported on inhibition of MBLs is based on the relatively rich literature developed during the design of inhibitors for zinc proteases and the strategies employed are closely aligned with the earlier protease inhibition studies.

Some of the inhibitors reported include:
trifluoromethyl alcohols and ketones (Walter, M. et al. 1996 “Trifluoromethyl Alcohol and Ketone Inhibitors of Metallo-β-Lactamases” Bioorg. Med. Chem. Lett., 6, 2455),
amino acid-derived hydroxamates (Walter, M. et al., 1990, Bioorg. Chem. 27, 35),
thiols (Bounaga, S., Laws, A. P., Galleni, M., Page, M. I. 1998, “The mechanism of catalysis and the inhibition of the Bacillus cereus zinc-dependent β-lactamase.” Biochem. J. 331, 703),
thioester derivatives (Hammond, G. G., Huber, J. L., Greenlee, M. L., Laub, J. B., Young, K., Silver, L. L., Balkovec, J. M., Pryor, K. D., Wu, J. K., Leiting, B., Pompliano, D. L., Toney, J. H. 1999 “Inhibition of IMP-1 metallo-β-lactamase and sensitization of IMP-1-producing bacteria by thioester derivatives” FEMS Microbiol. Lett. 179, 289-96),
cysteinyl peptides (Bounaga et al. 2001, “Cysteinyl peptide inhibitors of Bacillus cereus zinc β-lactamase”. Bioorg. Med. Chem. Lett. 11, 503),
biphenyl tetrazoles (Toney, J. H., Fitzgerald, P. M., Grover-Sharma, N., Olson, S. H., May, W. J., Sundelof, J. G., Vanderwall, D. E., Cleary, K. A., Grant, S. K., Wu, J. K., Kozarich, J. W., Pompliano, D. L., Hammond, G. G. “Antibiotic sensitization using biphenyl tetrazoles as potent inhibitors of Bacteroides fragilis metallo-β-lactamase.” Chem. Biol. 4, 185-96),
mercaptocarboxylates (Payne et al., 2000. “β-Lactamase epidemiology and the utility of established and novel beta-lactamase inhibitors.” Exp. Opin. Invest. Drugs 9, 247),
1-β-methylcarbapenem derivatives (Nagano, R., Adachi, Y., Hashizume, T., Morishima, H. 2000 “In vitro antibacterial activity and mechanism of action of J-111,225, a novel 1-β-methylcarbapenem, against transferable IMP-1 metallo-beta-lactamase producers.” J. Antimicrob. Chemother. 3, 271-6),
a synthetic cephamycin (Quiroga, M. I., Franceschini, N., Rossolini, G. M., Gutkind, G., Bonfiglio, G., Franchino, L., Amicosante, G. 2000 “Interaction of cefotetan and the metallo-β-lactamases produced in Aeromonas spp. and in vitro activity.” Chemotherapy, 46, 177),
2,3-disubstituted succinic acid derivatives (Toney, J. H., Hammond, G. G., Fitzgerald, P. M., Sharma, N., Balkovec, J. M., Rouen, G. P., Olson, S. H., Hammond, M. L., Greenlee, M. L., Gao, Y. D. 2001 “Succinic acids as potent inhibitors of plasmid-borne IMP-1 metallo-β-lactamase.” J. Biol. Chem. 276, 31913),
6-methylidene penems (Venkatesan A. M. et al. 2006 “Structure-Activity Relationship of 6-Methylidene Penems Bearing 6,5 Bicyclic Heterocycles as Broad-Spectrum β-Lactamase Inhibitors: Evidence for 1,4-Thiazepine Intermediates with C7 R Stereochemistry by Computational Methods” J. Med. Chem. 49, 4623-4637), and
6-mercaptomethyl-penicillanic acid sulfones (Buynak J. D. et al. 2004 “Penicillin-derived Inhibitors that simultaneously target both metallo- and serine-β-Lactamases” Bioorg. Med. Chem. Lett. 14, 1299-1304).
N-Sulfonyl hydrazones have also been reported to be inhibitors of the MBL IMP-1 (Siemann S., Evanoff D. P., Marrone L., Clarke A. J., Viswanatha T., Dmitrienko G. I. Antimicrob. Agents Chemother. 2002 “N-Arylsulfonyl Hydrazones as Inhibitors of IMP-1 Metallo-β-Lactamase. 46, 2450-7).

An interesting phage display strategy for identification of cysteinyl peptide inhibitors for MBLs has been reported recently by Levesque and coworkers (Sanschagrin, F., Levesque, R. C. 2005 “A specific peptide inhibitor of the class B metallo-β lactamase L-1 from Stenotrophomonas maltophilia identified using phage display” J. Antimicrob. Chemother. 55, 252-255). Despite these efforts, a practical MBL inhibitor with adequate potency, breadth of activity and suitable pharmacological properties has yet to be reported.

The literature concerning inhibition of the OXAs is relatively sparse compared to that associated with the other serine β-lactamases or MBLs (Georgopapadakou, N. 2004 “β-Lactamase inhibitors: evolving compounds for evolving resistance targets”, Expert Opin. Investig. Drugs, 13, 1307-18). Most OXAs are unaffected by the mechanism-based inhibitors (see FIG. 2) which are clinically useful for combating resistance caused by Class A β-lactamase producers. X-ray crystallographic studies by the groups of Mobashery and Samama (Golemi, D., Maveyraud, L., Vakulenko, S., Samama, J.-P., Mobashery, S. 2001 “Critical involvement of a carbamylated lysine in cartalytic function of class Dβ-lactamases” Proc. Natl. Acad. Sci., 98, 14280-14285), Strynadka (Paetzel, M., Danel, F., de Castro, L., Mossimann, S. C., Page, M. G. P. Strynadka, N. C. J. 2000 “Crystal structure of the class D β-lactamase OXA-10.” Nature Struct. Biol. 7, 918-925; Danel, F., Paetzel, M., Strynadka, N. C. J., Page, M. G. P. 2001 “Effect of Divalent Metal Cations on the Dimerization of OXA-10 and -14 Class D β-Lactamases from Pseudomonas aeruginosa” Biochemistry 40, 9412-9420), Knox (Sun, T., Nukaga, M., Mayama, K., Braswell, E. H., Knox, J. R. 2003 “Comparison of β-lactamases of classes A and D: 1.5-Å crystallographic structure of the class D OXA-1 oxacillinase” Protein Sci. 12, 82-91), and most recently by Santillana and co-workers (Santillana, E. et al. 2007 “Crystal structure of the carbapenemase OXA-24 reveals insights into the mechanism of carbapenem hydrolysis” Proc. Natl. Acad. Sci. USA 104, 5354-5359), have revealed a fascinating difference in mechanism of catalysis by the Class D enzymes relative to the other classes of serine-type β-lactamases. Whereas the Class A β-lactamases employ an active-site glutamate carboxylate group as a general base and the Class C enzymes employ a tyrosine side chain hydroxyl group, the Class D enzymes employ an N-carboxylysine residue formed by reaction of the active site lysine amino group with CO₂. Presumably it is this mechanistic difference that makes the mechanism-based inhibitors less effective against the Class D enzymes. Mugnier and co-workers reported that OXA-13 is inhibited by the carbapenem, imipenem (Mugnier, P., Podglajen, I., Goldstein, F. W., Collatz, E. 1998 “Carbapenems as inhibitors of OXA-13, a novel, integron-encoded β-lactamase in Pseudomonas aeruginosa.” Microbiology, 144, 1021-1031), and Mobashery and co-workers have demonstrated inhibition of OXA-10 by 6-hydroxyalkylpenicillanates. They have structurally characterized the inhibited enzyme as an acyl enzyme with the side chain hydroxyl group occupying the site where the water molecule involved in deacylation normally resides in the active site of the enzyme (Maveyraud, L., Golemi-Kotra, D., Ishiwata, A., Meroueh, O., Mobashery, S., Samama, J.-P. 2002 “High resolution X-ray structure of an acyl enzyme species for the Class D OXA-10β-lactamase” J. Am. Chem. Soc., 124, 2461-5).

Increasingly, however, the Class Dβ-lactamases that are being encountered in clinical settings are not inhibited by carbapenems but instead act as potent carbapenemases, rapidly destroying these antibiotics (Queenan A. M., Bush K. 2007 “Carbapenemases: the Versatile β-lactamases” Clin. Microbiol. Rev. 28, 440-458).

In light of the foregoing, there remains a need for broad-spectrum inhibitors for clinically important Class B metallo-β-lactamases (MBLs) and Class-D serine-type β-lactamases (the OXAs).

Compounds 1-5 of formula (I) below and their use as β-lactamase inhibitors were previously disclosed in an oral presentation by the inventors on May 30, 2006 at the 89th Canadian Chemistry Conference and Exhibition in Halifax, Nova Scotia, Canada. Compounds 1-3, 5, and 6-15 were also disclosed as β-lactamase inhibitors in an oral presentation by the inventors on Mar. 17, 2007 at the 35th Southern Ontario Undergraduate Student Chemistry Conference (SOUSCC) in Oshawa, Ontario, Canada, and in a poster presentation by the inventors on Jun. 3-7, 2007 at the 40th National Organic Symposium, Duke University, Durham, N.C., USA.

### SUMMARY OF THE INVENTION

The present invention provides inhibitors of β-lactamase enzymes. In one aspect, inhibitors of Class B metallo (MBL) and Class D (OXA) β-lactamases are provided. These β-lactamases currently render a growing number of bacterial strains resistant to the carbapenems, the β-lactam antibiotics of last resort, in treating antibiotic resistant infections especially in hospital settings.

More particularly, in one aspect there is provided a pharmaceutical composition useful for effecting β-lactamase inhibition in humans and animals which comprises a β-lactamase inhibitory amount of a compound of formula (I):

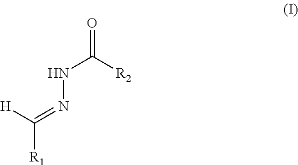

wherein
R₁ is selected from

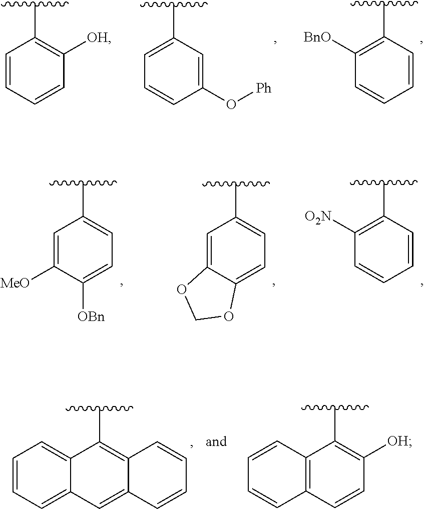

R₂ is selected from

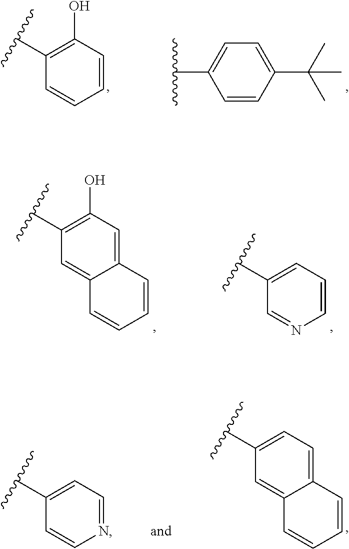

with the proviso that:
if R₁ is

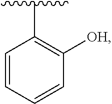

then R₂ is selected from

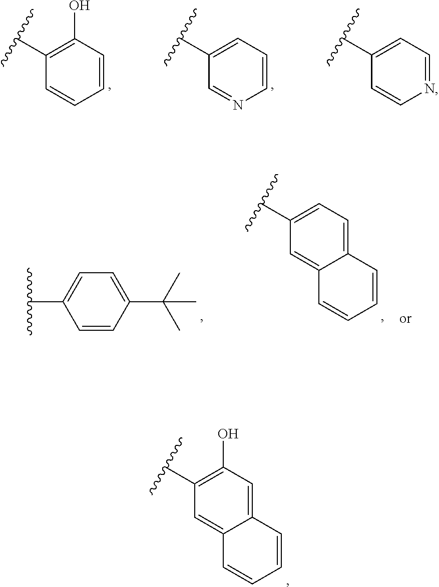

if R₁ is

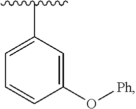

then R₂ is

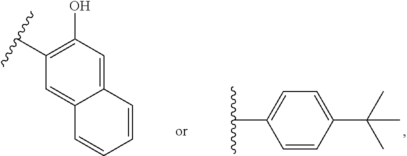

if R₁ is

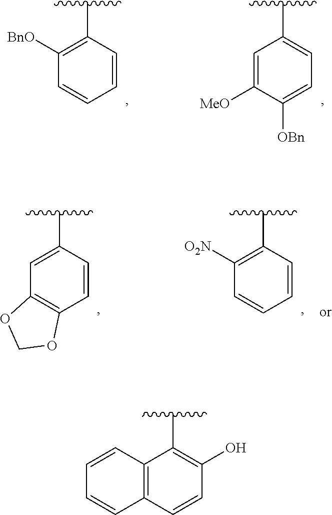

then R₂ is

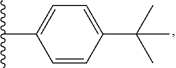

and
if R₁ is

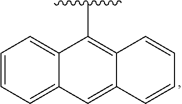

then R₂ is

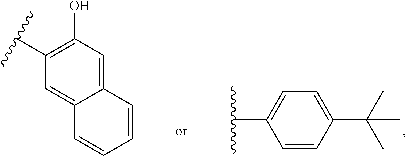

and a pharmaceutically acceptable carrier therefor.

In one aspect, said inhibition occurs in respect of at least one Class B or Class D β-lactamase enzyme.

In another aspect, the Class Bβ-lactamase enzyme is selected from IMP-1 and VIM-2.

In another aspect, the Class Dβ-lactamase enzyme is selected from OXA-10 and OXA-45.

The pharmaceutical compositions provided herein can be used in the manufacture of a medicament for the treatment of bacterial infections. In one aspect, the pharmaceutical compositions additionally comprise a pharmaceutically acceptable β-lactam antibiotic.

In another aspect, there is provided a method of treating a bacterial infection comprising administering to a mammalian patient in need of such treatment a compound of formula (I) as defined above in combination with a pharmaceutically acceptable β-lactam antibiotic in an amount which is effective for treating the bacterial infection. In one aspect, the bacterial infection comprises bacteria expressing at least one Class B or Class D β-lactamase enzyme.

Suitable β-lactam antibiotics may be selected from a penicillin, a cephalosporin, an oxacephem, a penem, or a carbapenem, for example. In one aspect, the β-lactam antibiotic is pipericillin.

In another aspect, the invention provides a method of inhibiting a β-lactamase enzyme, the method comprising contacting the β-lactamase enzyme with a compound of formula (I) as defined above. In yet another aspect, the β-lactamase enzyme is a Class B or Class D β-lactamase enzyme.

In another aspect, the compound of formula (I) is selected from:

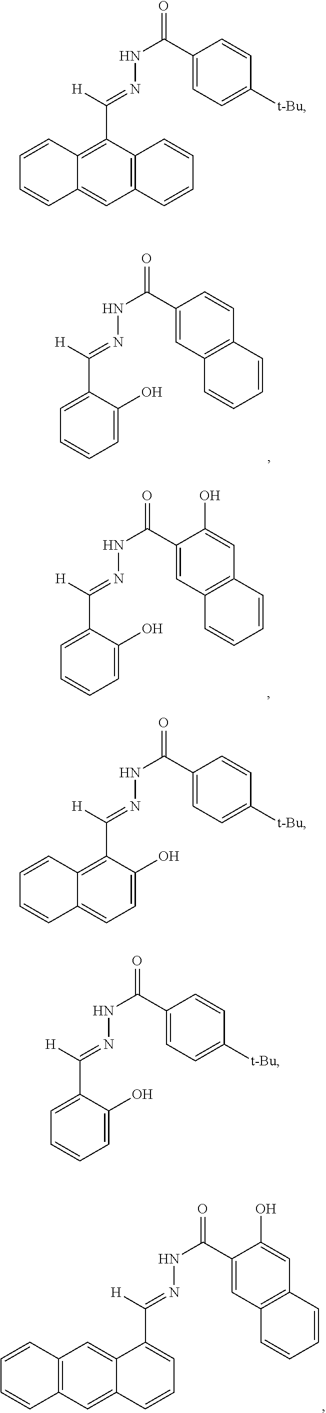

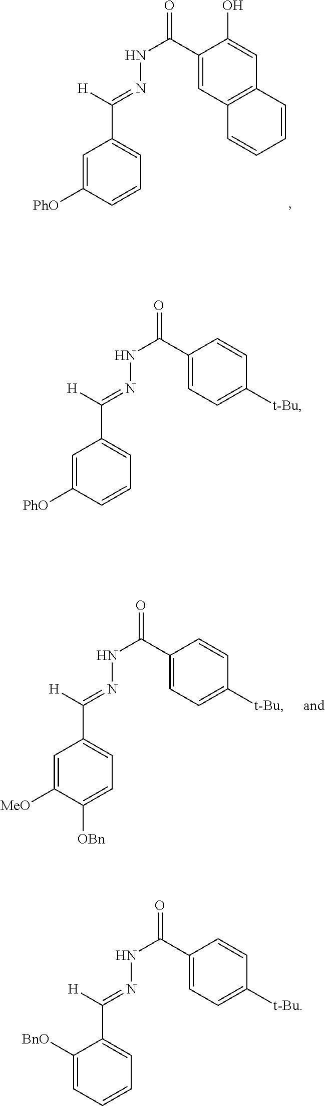

### BRIEF DESCRIPTION OF THE DRAWINGS

FIG. 1 illustrates the major structural classes of clinically useful β-lactam antibiotics.

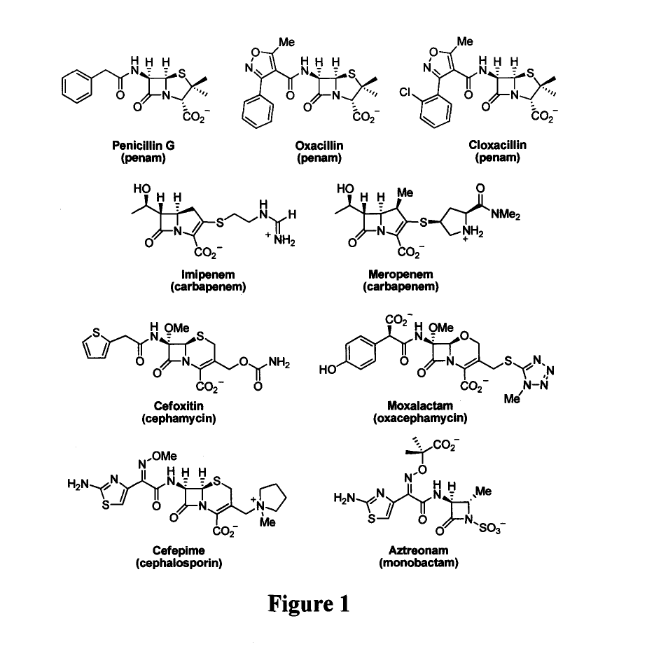

FIG. 2 illustrates some clinically important β-lactamase inhibitors.

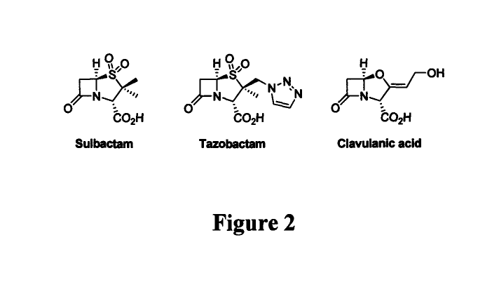

### DETAILED DESCRIPTION

In one embodiment, there is provided a pharmaceutical composition useful for effecting β-lactamase inhibition in humans and animals which comprises a β-lactamase inhibitory amount of a compound of formula (I):

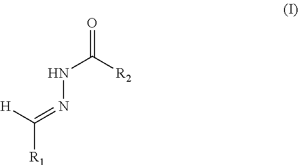

wherein
R₁ is selected from

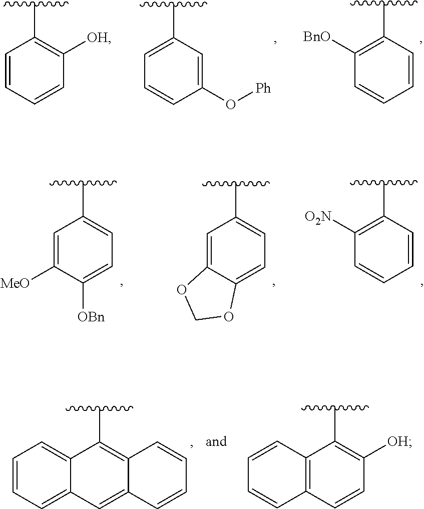

R₂ is selected from

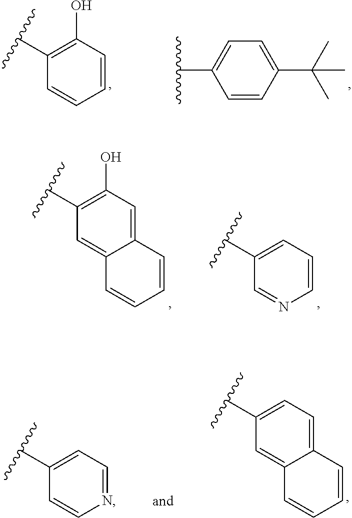

with the proviso that:
if R₁ is

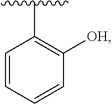

then R₂ is selected from

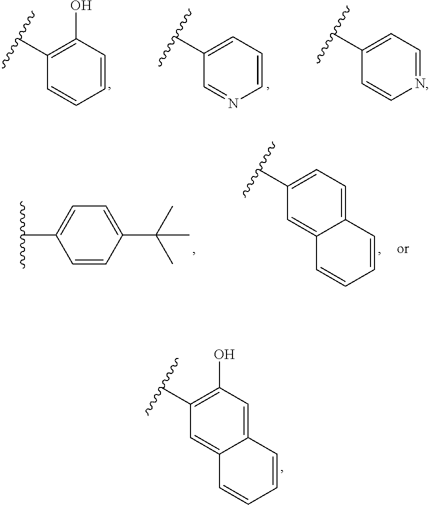

if R₁ is

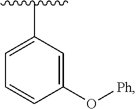

then R₂ is

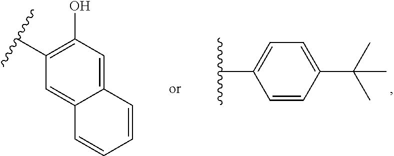

if R₁ is

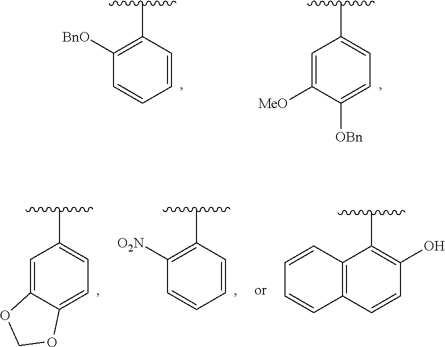

then R₂ is

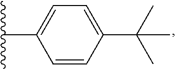

and
if R₁ is

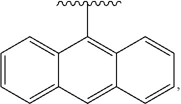

then R₂ is

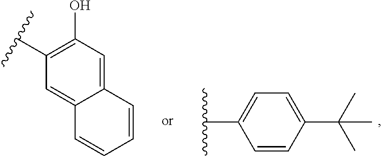

and a pharmaceutically acceptable carrier therefor.

The compounds disclosed herein were chosen for testing as β-lactamase inhibitors since these N-acylhydrazones possess an amide linkage within their structure which might mimic the β-lactam carbonyl oxygen atom of a normal β-lactam antibiotic substrate in binding to that active site of the target β-lactamases. The inventors have confirmed via structural studies that the conformational properties of N-acylhydrazones are significantly different than those of N-sulfonyl hydrazones previously reported as MBL inhibitors.

It is noted that many of the starting materials employed in the synthetic methods described herein are commercially available or are reported in the scientific literature. The compounds of formula (I) are known in the art and can be prepared as illustrated in Scheme 2 and as outlined in the Examples:

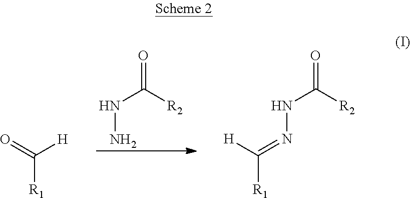

In general, warming an alcoholic solution of an aldehyde (R₁CHO) with an appropriate acylhydrazide (R₂(C═O)NHNH₂) followed by precipitation of the product assisted by the addition of water provides the subject compounds I in good yield and in a state of purity exceeding 95% as judged by TLC and ¹H NMR analysis.

The compounds of formula (I) can be formulated in pharmaceutical compositions by combining the compounds with a pharmaceutically acceptable carrier. Examples of such carriers are set forth below. The compounds of formula (I) have β-lactamase inhibitory properties, and are useful when combined with a β-lactam antibiotic for the treatment of infections in animals, especially mammals, including humans. The compounds may be used, for example, in the treatment of infections of the respiratory tract, urinary tract and soft tissues and blood, among others.

The compositions of the invention include those in a form adapted for administration by a variety of means: for instance, orally, topically, or parenterally by injection (such as intraveneously, intramuscularly, or subcutaneously). The compounds of formula (I) may be employed in powder or crystalline form, in liquid solution, or in suspension.

Suitable forms of the compositions of this invention include tablets, capsules, creams, syrups, suspensions, solutions, emulsions in oily or aqueous vehicles, reconstitutable powders and sterile forms suitable for injection or infusion. The pharmaceutical compositions may contain conventional pharmaceutically acceptable materials such as buffering agents, diluents, binders, colours, flavours, preservatives, disintegrants and the like in accordance with conventional pharmaceutical practice in the manner well understood by those skilled in the art of formulating antibiotics.

In injectable compositions, for instance, the carrier may be typically comprised of sterile water, saline, or another injectable liquid. Solutions of the compounds of formula (I) can be prepared in water, optionally mixed with a nontoxic surfactant. Dispersions can also be prepared in oils, and in glycerol, liquid polyethylene glycols, triacetin, and mixtures thereof. Under ordinary conditions of storage and use, these preparations typically contain a preservative to prevent the growth of microorganisms. Injectable solutions may be sterilized by incorporating the compound of formula (I) in the required amount in an appropriate solvent, with various other ingredients which may be desired, and filter sterilizing the resulting solution. Where sterile powders are needed, preferred methods of preparing these powders are vacuum drying and freeze-drying sterile solutions of the compounds of formula (I) in combination with other desired ingredients.

Topical compositions may be formulated in various carriers. Such carriers may be hydrophobic or hydrophilic bases to form ointments, creams, lotions, in aqueous, oleaginous or alcoholic liquids to form paints or in dry diluents to form powders. Useful solid carriers include finely divided solids such as talc, clay, microcrystalline cellulose, silica, alumina and the like. Useful liquid carriers include water, alcohols or glycols or water-alcohol/glycol blends, in which the compounds of formula (I) can be dissolved or dispersed at effective levels, optionally with the aid of non-toxic surfactants. Fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners known to those of skill in the art, such as synthetic polymers, fatty acids or salts and esters thereof, fatty alcohols, etc. can be used with liquid carriers to form spreadable pastes, gels, ointments, soaps, etc.

The following references disclose useful dermatological compositions which can be used to deliver the compounds of formula (I) to the skin: Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Oral compositions may be in the form of oral solutions or suspensions, or may be in tablet or capsule form (such as hard or soft shell gelatine capsules). Oral compositions include both extended release and immediate release delivery forms. Compositions for oral administration may also be incorporated directly with the food of a patient's diet. The compounds of formula (I) may be combined with one or more excipients and used in the form of ingestible tablets, buccal tablets, troches, capsules, elixirs, suspensions, syrups, wafers, and the like. Such compositions and preparations should contain at least 0.1% of active compound. The amount of the compounds of formula (I) in such therapeutically useful compositions is such that an effective dosage level will be obtained.

The above-mentioned compositions for oral administration may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, etc. may be added. When the unit dosage form is a capsule, it may contain a liquid carrier, such as a vegetable oil or a polyethylene glycol, in addition to materials of the above type. Various other materials may be present as coatings, etc. For instance, tablets, pills, or capsules may be coated with gelatin, wax, shellac or sugar and the like. A syrup or elixir may contain the active compound, a sweetening agent, one or more preservatives, a dye and a flavoring. Of course, any material used in preparing any unit dosage form should be pharmaceutically acceptable and substantially non-toxic in the amounts employed.

The compounds of formula (I) may be present in the composition as sole therapeutic agents or may be present together with other therapeutic agents such as a pharmaceutically acceptable β-lactam antibiotic. It is generally advantageous to use a compound of formula (I) in admixture or conjunction with a carbapenem, penicillin, cephalosporin or other β-lactam antibiotic or prodrug. It may also be advantageous to use a compound of formula (I) in combination with one or more β-lactam antibiotics, because of the β-lactamase inhibitory properties of the compounds. In this case, the compound of formula (I) and the β-lactam antibiotic can be administered separately or in the form of a single composition containing both active ingredients.

Thus, in one embodiment, there is provided a method of treating a bacterial infection comprising administering to a mammalian patient in need of such treatment a compound of formula (I) as defined above in combination with a pharmaceutically acceptable β-lactam antibiotic in an amount which is effective for treating the bacterial infection.

In another aspect, there is provided a method of inhibiting a β-lactamase enzyme, the method comprising contacting the β-lactamase enzyme with a compound of formula (I) as defined above.

Carbapenems, penicillins, cephalosporins, oxacephems, monobactums, penems, and other pharmaceutically acceptable β-lactam antibiotics suitable for co-administration with the compounds of Formula (I), whether by separate administration or by inclusion in the compositions according to the invention, include both those known to show instability to or to be otherwise susceptible to β-lactamases and also known to have a degree of resistance to β-lactamases. β-Lactam antibiotics which are well known in the art include those disclosed by R. B. Morin and M. Gorin, M. Eds.; Academic Press, New York, 1982; vol. 1-3, the contents of which are hereby incorporated herein by reference in this regard.

Examples of carbapenems that may be co-administered with the compounds of formula (I) include imipenem, meropenem, biapenem and doripenem (4R,5S,6S)-3-[3S,5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S,5R,6S)-2-(4-(2-[(((carbamoylmethyl)-1,4-diazoniabicyclo[2.2.2]oct-1-yl)-ethyl(1,8-naphthosultam)methyl)-6-[1(R)-hydroxyethyl]-1-methyl carbapen-2-em-3-carboxylate chloride, BMS181139 ([4R-[4alpha,5beta,6beta(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), BO2727 ([4R-3[3S*,5S*(R*)], 4alpha,5beta,6beta(R*)]]-6-(1-hydroxyethyl)-3-[154]-hydroxy-3-(methylamino)propyl]-3-pyrrolidinyl]thio]-4-methyl-7-oxo-1-azabicyclo[3.2.0]hept-2-ene-2-carboxylic acid monohydrochloride), E1010 ((1R,5S,6S)-6-[1(R)-hydroxymethyl]-2-[2(S)-[1(R)-hydroxy-1-[pyrrolidin-3(R)-yl]methyl]pyrrolidin-4(S)-ylsulfanyl]-1-methyl-1-carba-2-penem-3-carboxyl is acid hydrochloride), S4661((1R,5S,6S)-2-[(3S,5S)-5-(sulfamoylaminomethyl)pyrrolidin-3-yl]thio-6-[(1R)-1-hydroxyethyl]-1-methylcarbapen-2-em-3-carboxylic acid) and (1S,5R,6S)-1-methyl-2-{7-[4-(aminocarbonylmethyl)-1,4-diazoniabicyclo[2.2.2)octan-1yl]-methyl-fluoren-9-on-3-yl]-6-(1R-hydroxyethyl)-carbapen-2-em-3-carboxylate chloride and (+)-(4R,5S,6S)-6-[(1R)-1-1hydroxyethyl]-4-methyl-7-oxo-3[[3S,5S)-S-(sulfamoylaminomethyl)pyrrolidin-3-yl]thio]-1-azabicyclo[3.2.0]hept-2-ene-2-carboxylic acid monohydrate.

Certain carbapenems (e.g. imipenem) are susceptible to destruction by human renal dehydropeptidase (DHP); thus, pharmaceutical compositions comprising compounds of Formula (I) and such carbapenems may further comprise an inhibitor for DHP, such as cilastatin.

Examples of penicillins suitable for co-administration with the compounds of formula (I) include benzylpenicillin, phenoxymethylpenicillin, carbenicillin, azidocillin, propicillin, ampicillin, amoxycillin, epicillin, ticarcillin, cyclacillin, pirbenicillin, azlocillin, mezlocillin, sulbenicillin, piperacillin, and other known penicillins. The penicillins may be used in the form of pro-drugs thereof; for example as in vivo hydrolysable esters, for example the acetoxymethyl, pivaloyloxymethyl, α-ethoxycarbonyloxy-ethyl and phthalidyl esters of ampicillin, benzylpenicillin and amoxycillin; as aldehyde or ketone adducts of penicillins containing a 6-α-aminoacetamido side chain (for example hetacillin, metampicillin and analogous derivatives of amoxycillin); and as α-esters of carbenicillin and ticarcillin, for example the phenyl and indanyl α-esters.

Examples of cephalosporins that may be co-administered with the compounds of formula (I) include, cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftazidime, cefuroxime, cefinetazole, cefotaxime, ceftriaxone, and other known cephalosporins, all of which may be used in the form of pro-drugs thereof.

Examples of β-lactam antibiotics other than penicillins and cephalosporins that may be co-administered with the compounds of formula (I) include aztreonam, latamoxef (Moxalactam-trade mark), and other known β-lactam antibiotics such as carbapenems like imipenem, meropenem or (4R,5S,6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid, all of which may be used in the form of pro-drugs thereof.

Those of skill in the art will appreciate that useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula (I) in a liquid composition, such as a lotion, or a semi-solid or solid composition, such as a gel or a powder, will be from about 0.1-99 wt. %, preferably from about 0.5-25 wt. % for liquid compositions and 0.1-5 wt. % for semi-solid or solid compositions.

When the compositions according to this invention are presented in unit dosage form, each unit dose may suitably comprise from about 25 to about 1500 mg of a compound of formula (I), although lower or higher doses may be used in accordance with clinical practice. Appropriate dosages of the compounds of formula (I) may be readily ascertained by those of skill in the art.

When the compounds of formula (I) are co-administered with a penicillin, cephalosporin, carbapenem or other β-lactam antibiotic, the ratio of the amount of the compounds of formula (I) to the amount of the other β-lactam antibiotic may vary within a wide range.

The ratio may, for example, be from 1:100 to 100:1, 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:5 to 5:1, 1:4 to 4:1, 1:3 to 3:1, or 1:2 to 2:1. The ratio may also be 1:1. The amount of carbapenem, penicillin, cephalosporin or other β-lactam antibiotic will normally be approximately similar to the amount in which it is conventionally used.

Between 50 and 6000 mg of the compositions of the invention, for example, may be administered each day of treatment, although such daily dosages may be readily ascertained by those of skill in the art. For the treatment of severe systemic infections or infections of particularly intransigent organisms, higher doses may be used in accordance with clinical practice.

Compounds of the present application were evaluated as inhibitors of two Class B β-lactamases (IMP-1 and VIM-2) and two Class D β-lactamases OXA-10 and OXA-45).

IMP-1 was overexpressed in E. coli using the pCIP4 plasmid encoding IMP-1 which was obtained from Dr. M. Galleni, Université de Liège, B-4000 Liege, Belgium, and was purified as described by Laraki, N. et al., Antimicrob. Agents Chemother. 1999 43, 902-906.

VIM-2 was overexpressed in E. coli (DH10B) containing the pNOR,2001 plasmid, encoding VIM-2, which was provided by Dr. P. Nordmann, Universite de Paris, and was purified as described by Poirel et al., Antimicrob. Agents Chemother. 2000, 44, 891-897.

OXA-10 was overexpressed in E. coli using the pEt24a(+) plasmid, encoding OXA-10, which was provided by Dr. S. Mobashery, University of Notre Dame, and was purified as described by Golemi et al., J. Am. Chem. Soc. 2000, 122, 6132-6133.

OXA-45 was a gift from Dr. J. Spencer, University of Bristol, and had been prepared and purified as described by Toleman, M. A. et al., Antimicrob. Agents Chemother. 2003, 47, 2859-2863.

Two methods were used for measurement of β-lactamase activity, based on the use of the substrate nitrocefin. The first was carried out in a cuvette (final volume 1 ml, 25° C.) with the aid of a Cary 5 spectrophotometer, and second was with the use of a Molecular Devices Spectramax 190 96 well plate reader (final volume 100 μl, 30° C.).

All assays were performed in the appropriate buffer for the enzyme: (i) IMP-1, VIM-2: 50 mM Hepes pH 7.3, 1 μg/ml BSA, 1 μM ZnSO₄; (ii) OXA-45: 100 mM Na Phosphate/25 mM NaCO₃ pH 7.0, 1 μg/ml BSA; and (iii) OXA-10: 100 mM Na Phosphate/25 mM NaCO₃ pH 7.0. Enzyme concentrations were chosen so that a reasonable rate of reaction was observed. Inhibitors were dissolved in DMSO, with the final concentration of DMSO in the assay being 1%. Nitrocefin was dissolved in DMSO and diluted in 50 mM Hepes pH 7.3 with the final concentration in the assay being 100 μM. The enzyme was allowed to react with the inhibitor for a predetermined time followed by addition of substrate to initiate the reaction, which was monitored for an increase in absorbance at 482 nm. IC₅₀ values were determined by plotting percent loss of initial activity vs log inhibitor concentration. Controls were run to ensure that there was minimal loss of activity with DMSO (<10%). When inhibition by DMSO was noted, 0.5 M NaCl was added to stabilize the enzyme.

The results are summarized in Table 1.

TABLE 1

| | Compound: 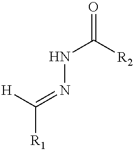 | IC50 (μM) class B class D | | | |
|---|---|---|---|---|---|
| No. | (I) | IMP-1 | VIM-2 | OXA-10 | OXA-45 |
| 1 | 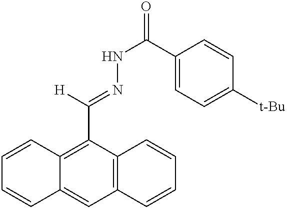 | 0.91 | | | |
| 2 | 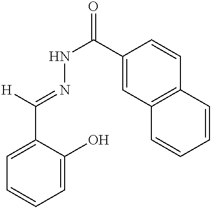 | 38.3 | 35.1 | 15.2 | 1.8 |
| 3 | 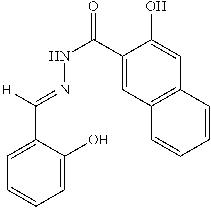 | 8.3 | 18.2 | 3.9 | 0.25 |
| 4 | 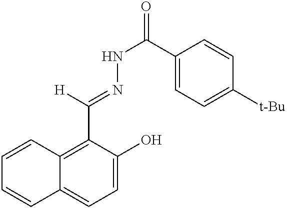 | 3 | 9 | 1.7 | 1.7 |
| 5 | 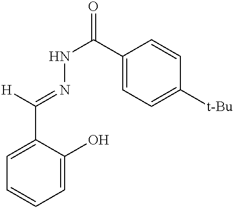 | 9 | 18 | 12.8 | 28 |
| 6 | 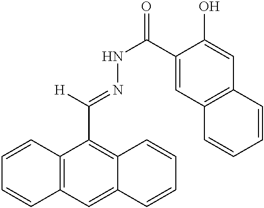 | 1.02 | 12.3 | 2.8 | 0.57 |
| 7 | 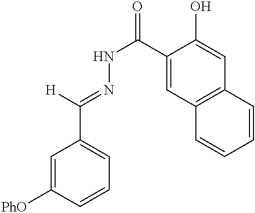 | 7.0 | 8.5 | 1.8 | 0.56 |
| 8 | 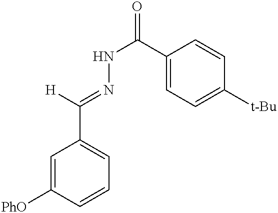 | 6.7 | 23.9 | 10.3 | 4.3 |
| 9 | 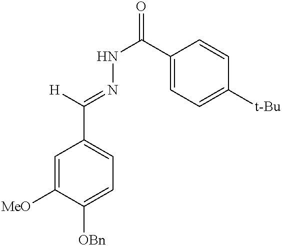 | 1.8 | >>10 | >>10 | >>10 |
| 10 | 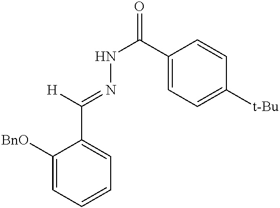 | 4.7 | >>10 | >>10 | >>10 |
| 11 | 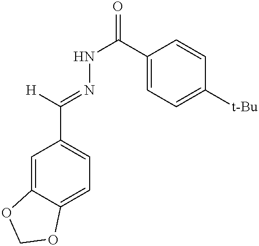 | >>10 | >>10 | >>10 | >>10 |
| 12 | 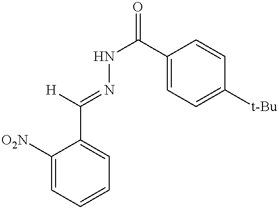 | >>10 | >>10 | >>10 | >>10 |
| 13 | 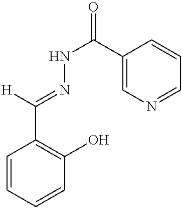 | >100 | >100 | >100 | >100 |
| 14 | 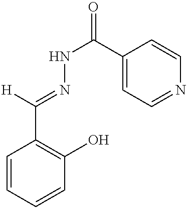 | >100 | >100 | >100 | >100 |
| 15 | 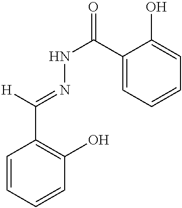 | >40 | >40 | >40 | >40 |

Molecular modeling studies involving computational docking of potential inhibitors into the active sites of IMP-1 and VIM-2, for which three dimensional structures have been reported based on protein crystallographic studies (pdb accession numbers 1DDK and PDB 1KO3 respectively), led the inventors to assess certain N-acylhydrazone compounds for affinity for the active sites of both of these metallo β-lactamases.

In particular, docking of N-acylhydrazones structures such that the oxygen atom of the acyl group acts as a ligand for the active site zinc ion in a fashion analogous to that expected for a β-lactam antibiotic substrate, revealed specific structural features of this structural class of molecules which would be expected to enhance their affinity for the active site of the enzymes and render them effective as inhibitors of β-lactam antibiotic hydrolysis.

Molecular modeling studies revealed that compound 1 can be docked computationally into the active site of IMP-1. The modeling study revealed that when R₁ is an extended polycyclic aromatic system, such as the anthracene group in 1, a favorable interaction of R₁ with the indole ring of a key tryptophan residue in the protein (Trp-28) was predicted to be enhanced.

The modeling study also revealed that a good fit of the N-acylhydrazone structure is achieved if R₂ is a benzene ring with a relatively large non-polar substituent in the para position of the aromatic ring which interacts with a specific hydrophobic pocket adjacent to the catalytic site of the enzyme.

Since it was of interest to discover inhibitors that also have activity against the clinically important MBL called VIM-2, the modeling exercise was extended by computationally superimposing the crystal structure of IMP-1 onto that of VIM-2 (pdb accession number PDB 1KO3) such that the actives sites coincided. Since VIM-2 does not have a tryptophan residue analogous to Trp-28 in IMP-1, the anthracene ring in 1, which was predicted to enhance the binding to IMP-1, was predicted to be less useful in enhancing binding to VIM-2.

Thus, N-acylhydrazones in which R₂ is a smaller aromatic ring system than the anthracene ring in 1 were considered.

Introduction of an ortho hydroxyl group in the aromatic ring R₁ was predicted to introduce an intramolecular H-bond with the hydrazone nitrogen thereby creating a more planar structure with the possibility of enhancing binding to the active site of VIM-2. Thus compound 2 to 5 which include such a hydroxyl group were appropriate for testing as potential inhibitors of both IMP-1 and VIM-2. In structures 2 and 3, the aromatic ring R₂ was chosen to probe the possibility that the bulky tertiary butyl substituent might be replaced by a naphthyl ring while still retaining some favorable interaction with the hydrophobic pocket in the active site. The ortho hydroxyl group on the naphthyl ring in 3 was predicted to potentially enhance binding by means of an H-binding interaction with an active site aspartic acid residue which is conserved in IMP-1, VIM-2 and other MBLs. The introduction of hydroxyl groups as in structures 2 to 5 was also expected to improve the water solubility relative to 1.

The Class D β-lactamases are typically referred to as oxacillinases (OXAs) because they are especially effective in the hydrolysis of the β-lactam bond in the penicillins known as the oxacillins (cloxacillin and oxacillin). The oxacillins possess a relatively large aromatic ring system in the amide side chain attached to C-6 of the penicillanic acid backbone. Computational superimposition of the N-acylhydrazone structures 2 to 5 onto a computed model of cloxacillin suggested that such compounds might bind to the active site of the oxacillinases in such a way as to allow the aromatic group R₂ to interact favourably with the hydrophobic binding pocket in the active site of the OXAs which allows these enzymes to bind the large aromatic side-chain of the oxacillins effectively. Thus, the compounds 2 to 5 were examined as potential inhibitors of the oxacillinases OXA-10 and OXA-45.

As shown above, compound 1 exhibited potent inhibition of IMP-1 (IC₅₀=0.9 μM), whereas compounds 2, 3, 4 and 5, which incorporate hydroxyl groups in R₁ and/or R₂ that decrease hydrophobicity and increases water solubility, inhibited not only the Class B β-lactamases (IMP-1 and VIM-2) but also the Class D β-lactamases OXA-10 and OXA-45).

In the case of docking to the active site of IMP-1, as noted above, it was observed that for models in which R₁ is an extended aromatic system such as 9-anthracenyl, favourable contacts were achieved between such a ring and the indole ring of tryptophan-28 (referred to here as binding region A). Since VIM-2 does not possess such a tryptophan residue near the active site, other potential structural features of an N-acyl hydrazone compound which might create favourable interactions with the enzyme were considered. The observation of a hydrophobic pocket in the active site of VIM-2 (involving the side chains of amino acid residues Trp-87) and a similar structural feature in the active site of IMP-1 (involving amino acid residues Phe-51) (referred to here as binding region B) led to the consideration that incorporating a 2-naphthyl group as R₂ might enhance the binding of an N-acylhydrazone to both IMP-1 and VIM-2. Furthermore, the computational model suggested that placing a hydroxyl group ortho to the acyl group in R₂ could lead to a favourable hydrogen bonding interaction with the side-chain carboxylate group of Asp-81 in IMP-1 and Asp-120 in VIM-2 (referred to here as binding region C) which is present in both enzymes and appears to be conserved among all MBLs studied to date. Thus, compound 6 which incorporates all of these structural features was chosen for enzyme inhibition studies.

Additionally, the molecular modeling study suggested that placement of a phenoxy substituent in the meta position of a benzene ring functioning as R₁ might lead to a favorable interaction with another hydrophobic region of the active site defined by amino acids Val-25 and Val-31 in IMP-1 and a related site defined by amino acid Phe-61 in VIM-2 (referred to here as binding region D). Thus, N-acylhydrazone structure 7 which possesses structural features predicted to provide affinity for binding regions B, C and D in IMP-1 and in VIM-2 was chosen for enzyme inhibition studies. Likewise, compound 8, which possesses a meta-phenoxy group as R₁ predicted to interact favorably with binding region D and a p-t-butylphenyl group as R₂, predicted to interact favorably with binding region C, was also studied.

The molecular modeling study also predicted that an aromatic ring of an ortho benzyloxy group in R₁ might interact favorably with binding region A which is present in IMP-1 but not present in VIM-2. Thus compound 10 was also chosen for study.

Compound 9, which features a para benzyloxy group in R₁ with potential for favorable interaction with binding region A and a para t-butyl group in R₂ capable of interacting favorably with binding region B was also examined as a potential inhibitor.

Compounds 11 and 12, which possess a para t-butyl group in R₂ capable of favorable interaction with binding region B, but lack functionality which would be predicted to provide favorable interactions with the other binding regions, were also examined to probe the validity of the binding model.

Likewise compounds 13 and 14 which lack groups appropriate for favorable interactions with binding regions B, C or D were examined to test the model.

In addition compound 15, which possesses a group in R₂ for favorable interaction with binding region C but lacking functionality for strong interaction with binding regions A, B and D was also examined.

Compounds 6-15 were examined as potential inhibitors of the oxacillinases OXA-10 and OXA-45. Compounds 6-8 were found to be good inhibitors of the Class D β-lactamases OXA-10 and OXA-45.

Such compounds were expected to enhance the potency of a clinically useful β-lactam antibiotic against clinical isolates of human pathogenic bacteria which are highly resistant to β-lactam antibiotics. Certain β-lactamase inhibitors were tested to determine whether or not they would have a synergistic effect on the antibacterial activity of pipericillin against β-lactam antibiotic resistant bacterial clinical isolates using the Agar Dilution Method.

Bacterial colonies were taken from overnight blood agar plates, and sterile saline was inoculated to make a 0.5 McFarland suspension inoculum and further diluted such that the 10⁴ CFU spots were delivered to the MHA (Oxoid) surface. Inhibitor samples were dissolved in DMSO and administered at a pipericillin:compound ratio of 4:1 (wt/wt). The plates were incubated at 35° C. and read after 18 h of incubation. MICs were measured by agar dilution on Mueller-Hinton II agar (BD Microbiology Systems, Cockeysville, Md., USA), as recommended by the NCCLS (National Committee for Clinical Laboratory Standards. (2003).Performance Standards for Antimicrobial Susceptibility Testing.)

Compound 5 was capable of causing a diminution in the MIC value found for the clinically important penicillin, pipericillin, in microbiological plate assays, against a highly resistant strain (92-00626) of Stenotrophomonas maltophilia which produces the metallo β-lactamase known as L1, by greater than 4 fold at a concentration of 5 which is ¼ that of the antibiotic. Additionally, compound 3 was capable of causing a diminution in the MIC value found for the clinically important penicillin, pipericillin, in microbiological plate assays, against a highly resistant strain (81-11963) of Pseudomonas aeruginosa which produces the metallo β-lactamase known as VIM-2, by greater than 4 fold at a concentration of 3 which is ¼ that of the antibiotic.

### EXAMPLES

### Experimental Procedures

### General

All chemical reagents were purchased from Alfa Aesar or Sigma-Aldrich and were used as supplied. Solvents were distilled prior to use. ¹H NMR and ¹³C NMR spectra were recorded on Brüker AC-300, AVANCE 300, and AVANCE 500 spectrometers. Chemical shifts in ¹H NMR and ¹³C NMR spectra are reported in parts per million (ppm) relative to tetramethylsilane (TMS), with calibration of the residual solvent peaks according to values reported by Gottlieb et al. (J. Org. Chem. 1997, 62, 7512-7515). When peak multiplicities are given, the following abbreviations are used: s, singlet; d, doublet; t, triplet; q, quartet; sept., septet; dd, doublet of doublets; m, multiplet; br, broad; app., apparent; gem, geminal.

Although two or more conformations were detected in the NMR spectra of several N-acyl hydrazones, only the signals for the major conformers are reported.

Each of the aldehydes and benzhydrazides employed in this study are known and commercially available.

### Hydrazone Condensations

### General Procedure

The aldehyde (0.5-5.0 mmol) was combined with the appropriate benzhydrazide (1.0±0.1 equiv.) and suspended in absolute ethanol (3-4 mL/mmol). The mixture was lowered into a hot oil bath and stirred at 70° C. for 2-24 h. Typically the reaction mixtures cleared upon heating and many of the hydrazone condensation products precipitated to a considerable extent as the reaction proceeded. When the condensations were judged to be complete, the mixture was cooled to 0° C. and additional precipitation was induced by the addition of cold water (1-2 volumes). The resulting solid was filtered and rinsed with cold water (3×1 mL).

### N′-(Anthracen-9-ylmethylene)-4-t-butylbenzohydrazide (1)

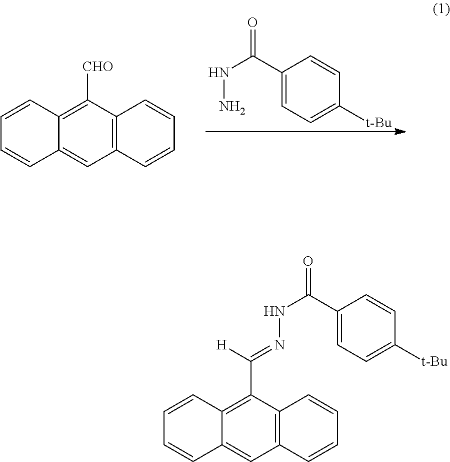

This material is known (CAS 328921-44-4) and commercially available but was prepared according to the general procedure given above. Condensation of 9-anthraldehyde (207 mg, 1.00 mmol) and t-butylbenzhydrazide (195 mg, 1.02 mmol) provided the title compound as a yellow solid (353.0 mg, 92%). ¹H NMR (300 MHz, DMSO-d₆): δ 1.34 (s, 9H), 7.53-7.68 (m, 6H), 7.96 (d, J=8.0 Hz, 2H), 8.16 (d, J=8.3 Hz, 2H), 8.73 (s, 1H), 8.76 (d, J=8.3 Hz, 2H), 9.66 (s, 1H), 12.03 (s, 1H).

### N′-(2-Hydroxybenzylidene)-2-naphthohydrazide (2)

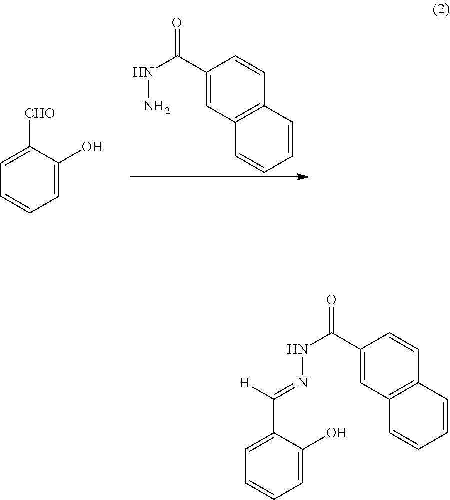

This material is known (CAS 358393-73-4; Sacconi, L. J. Am. Chem. Soc. 1954, 76, 3400-3402) and commercially available but was prepared according to the general procedure given above. Condensation of salicylaldehyde (66.8 mg, 0.547 mmol) and 2-naphthoic hydrazide (101 mg, 0.54 mmol) provided the title compound as a white solid (118 mg, 75%). ¹H NMR (300 MHz, DMSO-d₆): δ 6.90-6.95 (m, 2H), 7.30 (t, J=7.0 Hz, 1H), 7.53-7.67 (m, 3H), 7.96-8.10 (m, 4H), 8.56 (s, 1H), 8.69 (s, 1H), 11.30 (s, 1H), 12.27 (s, 1H).

### 3-Hydroxy-N′-(2-hydroxybenzylidene)-2-naphthohydrazide (3)

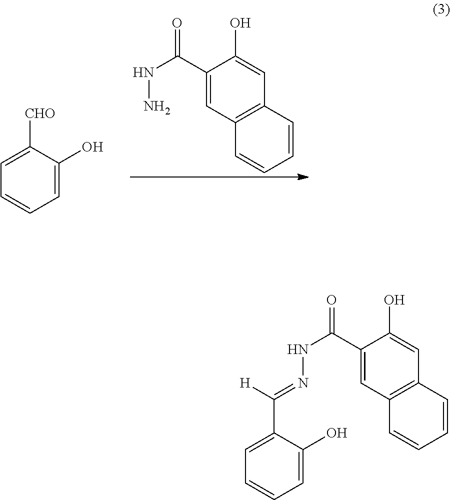

This material is known (CAS 80648-84-6, 854616-45-8; Buu-Hoi, N. P.; Xuong, N. D.; Nam, N. H.; Binon, F.; Royer, R. J. Chem. Soc. 1953, 1358-1364) and commercially available but was prepared according to the general procedure given above. Condensation of salicylaldehyde (200 mg, 1.64 mmol) and 3-hydroxy-2-naphthoic hydrazide (300 mg, 1.49 mmol) provided the title compound as a beige solid (430.5 mg, 95%). ¹H NMR (300 MHz, DMSO-d₆): δ 6.87-6.95 (m, 2H), 7.26-7.36 (m, 3H), 7.48 (t, J=7.4 Hz, 1H), 7.55 (d, J=7.4 Hz, 1H), 7.73 (d, J=8.3 Hz, 1H), 7.88 (d, J=8.2 Hz, 1H), 8.43 (s, 1H), 8.65 (s, 1H), 11.19 (s, 1H), 11.2 (br s, 1H), 12.1 (br s, 1H). LRMS (EI) m/z (relative intensity): 306 (M⁺, 70), 171 (100), 115 (30). HRMS (EI) m/z: 306.1004 calculated for C₁₈H₁₄N₂O₃; 306.1002 observed.

### 4-t-Butyl-N′-((2-hydroxynaphthalen-1-yl)methylene)benzohydrazide (4)

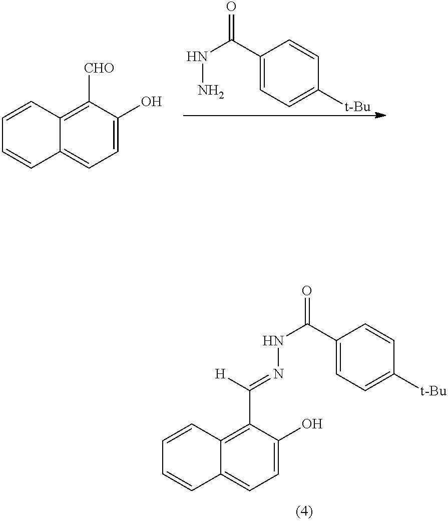

This material is known (CAS 68758-85-0; Meink, P.; Leroux, V.; Sergheraert, C.; Grellier, P. Bioorg. Med. Chem. Lett. 2006, 16, 31-35) and commercially available but was prepared according to the general procedure given above. Condensation of 2-methoxy-1-naphthaldehyde (308 mg, 1.79 mmol) and 3-hydroxy-2-naphthoic hydrazide (345 mg, 1.79 mmol) provided the title compound as a dark yellow solid (538 mg, 87%).

¹H NMR (300 MHz, DMSO-d₆): δ 1.32 (s, 9H), 7.23 (d, J=9.0 Hz, 1H), 7.41 (t, J=7.4 Hz, 1H), 7.55-7.65 (m, 314), 7.83-7.96 (m, 4H), 8.21 (d, J=8.5 Hz, 1H), 9.48 (s, 1H), 12.1 (br s, 1H).

### 4-t-Butyl-N′-(2-hydroxybenzylidene)benzohydrazide (5)

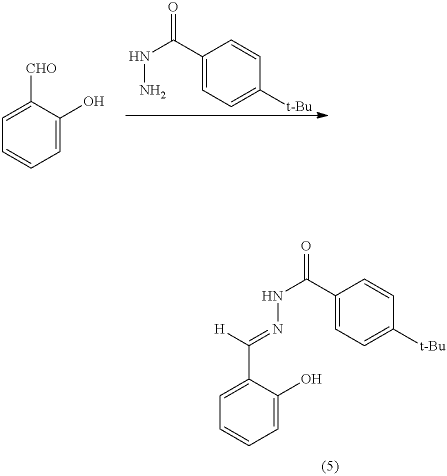

This material is known (CAS 82859-75-4, 159597-78-1; Melnk, P.; Leroux, V.; Sergheraert, C.; Grellier, P. Bioorg. Med. Chem. Lett. 2006, 16, 31-35) and commercially available but was prepared according to the general procedure given above. Condensation of salicylaldehyde (109 mg, 0.89 mmol) and 4-t-butylbenzhydrazide (172 mg, 0.90 mmol) provided the title compound as a white powder (238 mg, 90%). ¹H NMR (300 MHz, DMSO-d₆): δ 1.31 (s, 9H), 6.88-6.95 (m, 2H), 7.29 (t, J=7.2 Hz, 1H), 7.50-7.58 (m, 1H), 7.55 (d, J=8.3 Hz, 2H), 7.87 (d, J=8.3 Hz, 2H), 8.62 (s, 1H), 11.3 (br s, 1H), 12.0 (br s, 1H).

### N′-(Anthracen-9-ylmethylene)-3-hydroxy-2-naphthohydrazide (6)

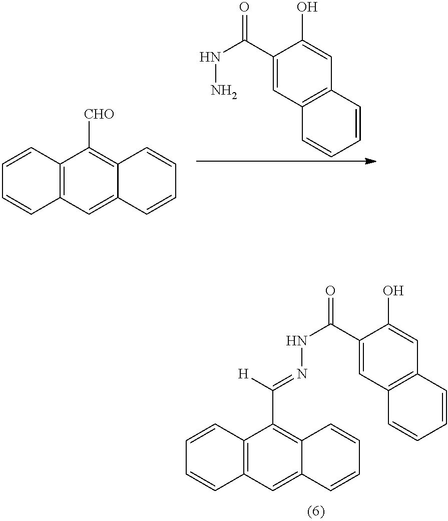

This material is known (CAS 91989-63-8; Enomoto, K.; Ito, A. Japanese Patent 61022346, 1986) and commercially available but was prepared according to the general procedure given above. Condensation of 9-anthraldehyde (112.9 mg, 0.547 mmol) and 3-hydroxy-2-naphthoic hydrazide (100.3 mg, 0.496 mmol) provided the title compound as a bright yellow powder (174.7 mg, 90%). ¹H NMR (300 MHz, DMSO-d₆): δ 7.25-7.77 (m, 7H), 7.79 (d, J=8.3 Hz, 1H), 7.97 (d, J=8.3 Hz, 1H), 8.17 (d, J=8.2 Hz, 2H), 8.76 (s, 1H), 8.80 (d, J=8.8 Hz, 2H), 9.69 (s, 1H), 11.4 (br s, 1H), 12.3 (br s, 1H). LRMS (EI) m/z (relative intensity): 390 (M⁺, 15), 219 (10), 203 (100), 171 (20). HRMS (EI) m/z: 390.1368 calculated for C₂₆H₁₈N₂O₂; 390.1371 observed.

### 3-Hydroxy-N-(3-phenoxybenzylidene)-2-naphthohydrazide (7)

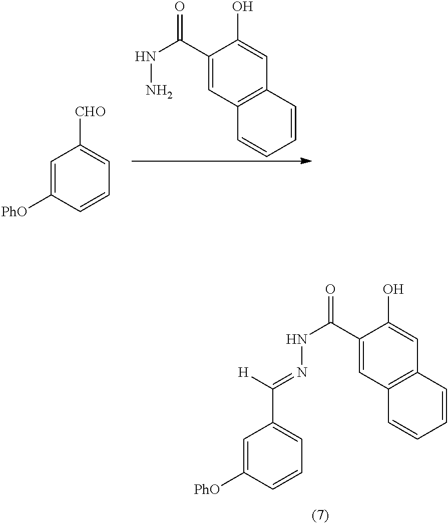

This material is known (CAS 351214-95-4) and commercially available but was prepared according to the general procedure given above. Condensation of 3-phenoxybenzaldehyde (111.1 mg, 0.5605 mmol) and 3-hydroxy-2-naphthoic hydrazide (103.0 mg, 0.5094 mmol) provided the title compound as a beige solid (170.3 mg, 87%). ¹H NMR (300 MHz, DMSO-d₆): δ 7.05 (d, J=8.1 Hz, 2H), 7.07-7.12 (m, 1H), 7.16 (t, J=7.4 Hz, 1H), 7.28-7.52 (m, 8H), 7.72 (d, J=8.3 Hz, 1H), 7.87 (d, J=8.1 Hz, 1H), 8.39 (s, 1H), 8.41 (s, 1H), 11.4 (br s, 1H), 11.9 (br s, 1H). LRMS (EI) m/z (relative intensity): 382 (M⁺, 65), 171 (100), 115 (35). HRMS (EI) m/z: 382.1317 calculated for C₂₄H₁₈N₂O₃; 382.1310 observed.

### 4-t-Butyl-N′-(3-phenoxybenzylidene)benzohydrazide (8)

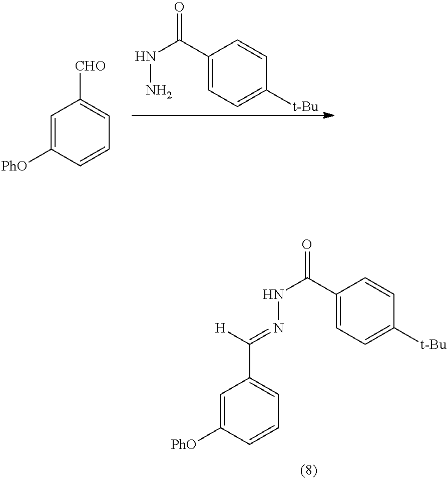

This material is known (CAS 611196-49-7) and commercially available but was prepared according to the general procedure given above. Condensation of 3-phenoxybenzaldehyde (115.7 mg, 0.5837 mmol) and 4-t-butylbenzhydrazide (101.0 mg, 0.5254 mmol) provided the title compound as a white solid (167.2 mg, 86%). ¹H NMR (300 MHz, DMSO-d₆): δ 1.29 (s, 9H), 7.06 (m, 3H) 7.15 (t, J=7.4 Hz, 1H), 7.33 (s, 1H), 7.38-7.47 (m, 4H), 7.52 (d, J=8.0 Hz, 2H), 7.82 (d, J=8.0, 2H), 8.42 (s, 1H), 11.79 (s, 1H). LRMS (EI) m/z (relative intensity): 372 (M⁺, 10), 195 (40), 161 (100), 118 (10). HRMS (EI) m/z: 372.1838 calculated for C₂₄H₂₄N₂O₂; 372.1829 observed.

### N′-(4-(Benzyloxy)-3-methoxybenzylidene)-44-butylbenzohydrazide (9)

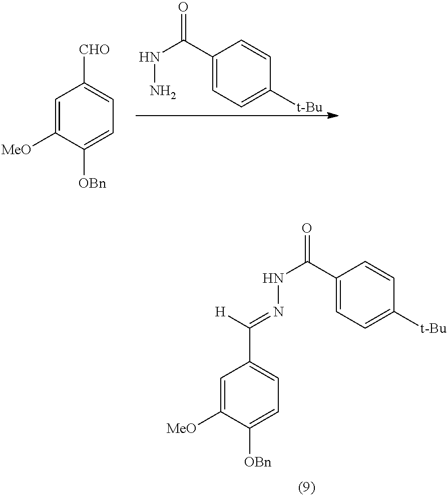

This material is known (CAS 300672-15-5) and commercially available but was prepared according to the general procedure given above. Condensation of 4-benzyloxy-3-methoxybenzaldehyde (139.8 mg, 0.5770 mmol) and 4-t-butylbenzhydrazide (100.8 mg, 0.5243 mmol) provided the title compound as a beige solid (194.3 mg, 89%). ¹H NMR (300 MHz, DMSO-d₆): δ 1.31 (s, 9H), 3.83 (s, 3H), 5.13 (s, 2H), 7.08-7.19 (m, 2H), 7.31-7.47 (m, 6H), 7.53 (d, J=8.4 Hz, 2H), 7.83 (d, J=8.3 Hz, 2H), 8.37 (s, 1H), 11.65 (s, 1H).

### N′-(2-(Benzyloxy)benzylidene)-4-t-butylbenzohydrazide (10)

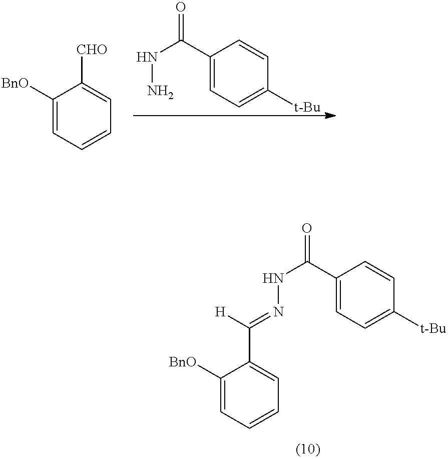

This material is known (CAS 514810-69-6) and commercially available but was prepared according to the general procedure given above. Condensation of 2-benzyloxybenzaldehyde (122.0 mg, 0.5748 mmol) and 4-t-butylbenzhydrazide (100.3 mg, 0.5217 mmol) provided the title compound as a white powder (174.9 mg, 87%). ¹H NMR (300 MHz, DMSO-d₆): δ 1.30 (s, 9H), 5.20 (s, 2H), 7.03 (t, J=7.5 Hz, 1H), 7.21 (d, J=8.3 Hz, 1H), 7.19-7.44 (m, 4H), 7.49-7.53 (m, 4H), 7.83 (d, J=8.2 Hz, 2H), 7.90 (d, J=7.5 Hz, 1H), 8.81 (s, 1H), 11.82 (s, 1H). LRMS (EI) m/z (relative intensity): 386 (M⁺, 4), 268 (15), 195 (30), 161 (100). HRMS (EI) m/z: 386.1994 calculated for C₂₅H₂₆N₂O₂; 386.1989 observed.

### N′-(Benzo[d][1,3]dioxol-5-ylmethylene)-4-t-butylbenzohydrazide (11)

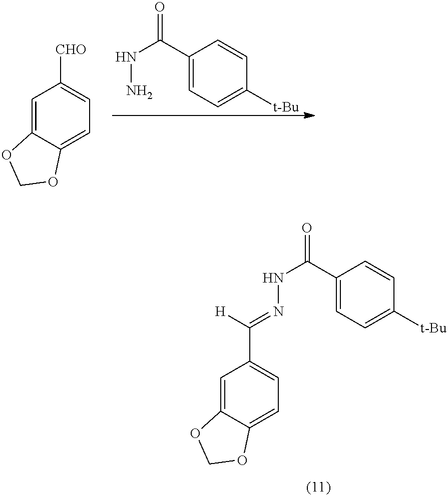

This material is known (CAS 326923-85-7) and commercially available but was prepared according to the general procedure given above. Condensation of piperonal (90.7 mg, 0.604 mmol) and 4-t-butylbenzhydrazide (101.1 mg, 0.526 mmol) provided the title compound as a white powder (145.2 mg, 85%). ¹H NMR (300 MHz, DMSO-d₆): δ 1.31 (s, 9H), 6.08 (s, 2H), 6.98 (d, J=7.9 Hz, 1H), 7.16 (d, J=7.9 Hz, 1H), 7.29 (s, 1H), 7.52 (d, J=8.3 Hz, 2H), 7.83 (d, J=8.3 Hz, 2H), 8.35 (s, 1H), 11.66 (s, 1H). LRMS (EI) m/z (relative intensity): 324 (M⁺, 30), 178 (20), 161 (100). HRMS (EI) m/z: 324.1474 calculated for C₁₉H₂₀N₂O₃; 324.1470 observed.

### 4-t-Butyl-N′-(2-nitrobenzylidene)benzohydrazide (12)

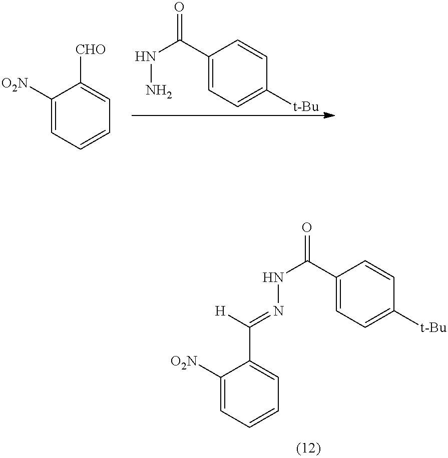

This material is known (CAS 328921-27-3) and commercially available but was prepared according to the general procedure given above. Condensation of 2-nitrobenzaldehyde (95.6 mg, 0.633 mmol) and 4-t-butylbenzhydrazide (104.9 mg, 0.546 mmol) provided the title compound as an off-white powder (143.0 mg, 81%). ¹H NMR (300 MHz, DMSO-d₆): δ 1.31 (s, 9H), 7.55 (d, J=8.3 Hz, 2H), 7.67 (t, J=7.3 Hz, 1H), 7.78-7.90 (m, 3H), 8.07 (d, J=8.1 Hz, 1H), 8.13 (br d, J=7.3 Hz, 1H), 8.86 (br s, 1H), 12.12 (br s, 1H). LRMS (EI) m/z (relative intensity): 325 (M⁺, 2), 177 (15), 161 (100). HRMS (EI) m/z: 325.1426 calculated for C₁₈H₁₉N₃O₃; 325.1421 observed.

### N′-(2-Hydroxybenzylidene)nicotinohydrazide (13)

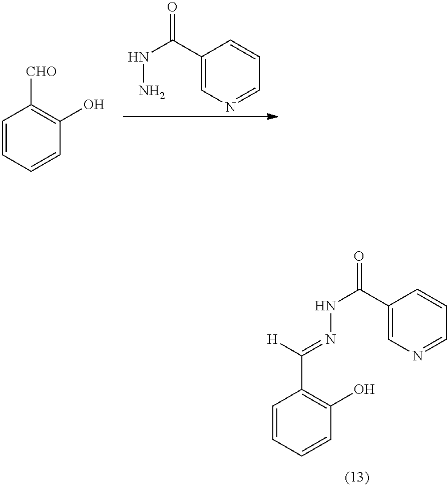

This material is known (CAS 15017-28-4, 71112-97-5, Melnk, P.; Leroux, V.; Sergheraert, C.; Grellier, P. Bioorg. Med. Chem. Lea. 2006, 16, 31-35) and commercially available but was prepared according to the general procedure given above. Condensation of salicylaldehyde (115.9 mg, 0.0949 mmol) and nicotinic hydrazide (111.6 mg, 0.814 mmol) provided the title compound as a light yellow powder (131.6 mg, 67%). ¹H NMR (300 MHz, DMSO-d₆): δ 6.90-6.95 (m, 2H), 7.30 (dt, J=7.1 Hz, J=1.6 Hz, 1H), 7.56-7.60 (m, 2H), 8.27 (d, J=8.0 Hz, 1H), 8.64 (s, 1H), 8.77 (br s, 1H), 9.08 (s, 1H), 11.13 (s, 1H), 12.24 (s, 1H). ¹³C NMR (125 MHz, DMSO-d₆): δ 116.9, 119.2, 119.9, 124.1, 129.2, 129.8, 132.1, 135.9, 149.0, 149.1, 152.9, 157.9, 161.9.

### N′-(2-Hydroxybenzylidene)isonicotinohydrazide (14)

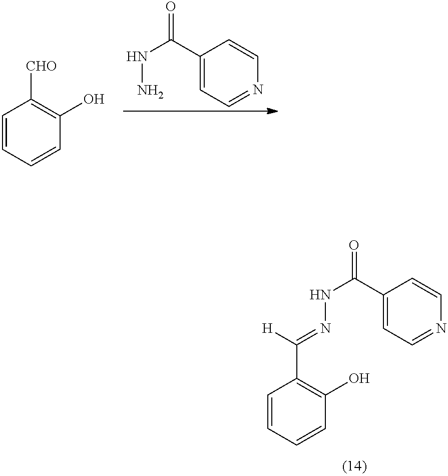

This material is known (CAS 495-84-1, 263153-48-6, Ponka, P.; Schulman, H. M. J. Biol. Chem. 1985, 260, 14717-14721) and commercially available but was prepared according to the general procedure given above. Condensation of salicylaldehyde (119.0 mg, 0.975 mmol) and isoniazid (113.5 mg, 0.828 mmol) provided the title compound as a white powder (103.2 mg, 52%). ¹H NMR (300 MHz, DMSO-d₆): δ 6.90-6.94 (m, 2H), 7.31 (t, J=6.8 Hz, 1H), 7.59-7.60 (m, 1H), 7.84 (dd, J=1.6 Hz, J=4.4 Hz 2H), 8.67 (s, 1H), 8.79 (m, 2H), 11.06 (s, 1H), 12.28 (br s, 1H).

### 2-Hydroxy-N′-(2-hydroxybenzylidene)benzohydrazide (15)

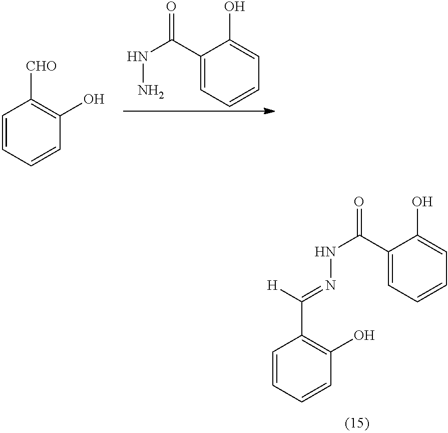

This material is known (CAS 3232-36-8, 191848-55-2, Buu-Hoi, N. P.; Xuong, N. D.; Nam, N. H.; Binon, F.; Royer, R. J. Chem. Soc. 1953, 1358-1364) and commercially available but was prepared according to the general procedure given above. Condensation of salicylaldehyde (95.2 mg, 0.780 mmol) and 2-hydroxybenzhydrazide (106.5 mg, 0.670 mmol) provided the title compound as a light yellow powder (159.0 mg, 89%). ¹H NMR (300 MHz, DMSO-d₆): δ 6.76-6.96 (m, 4H), 7.28 (t, J=8.2 Hz, 1H), 7.42 (t, J=8.3 Hz, 1H), 7.53 (d, J=7.1 Hz, 1H), 7.86 (d, J=7.7 Hz, 1H), 8.64 (s, 1H), 11.16 (s, 1H), 11.8 (br s, 1H), 12.0 (br s, 1H).

It will be understood that numerous modifications thereto will appear to those skilled in the art. Accordingly, the above description should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

The embodiments of the invention described above are intended to be exemplary only. The scope of the invention is therefore intended to be limited solely by the scope of the appended claims.

Every reference cited herein is hereby incorporated by reference in its entirety.

We claim:

1. A pharmaceutical composition useful for effecting β-lactamase inhibition in humans and animals which comprises a β-lactamase inhibitory amount of a compound of formula (I): 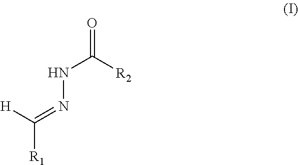
wherein
R₁ is selected from 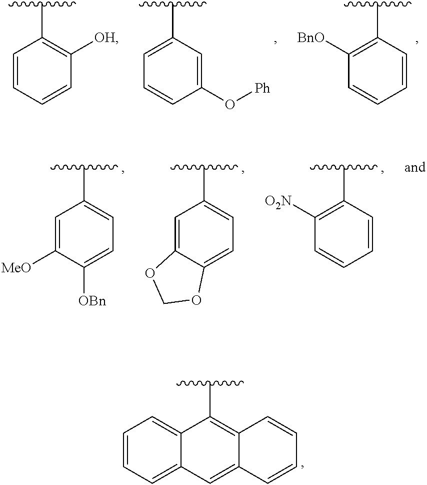
R₂ is selected from 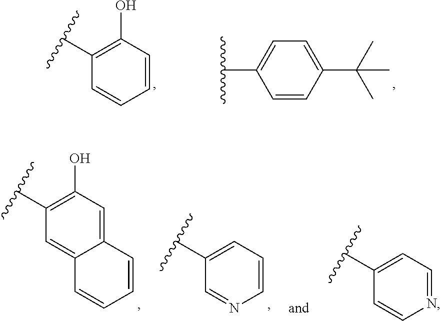
with the proviso that:
if R₁ is 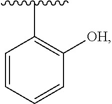
then R₂ is selected from 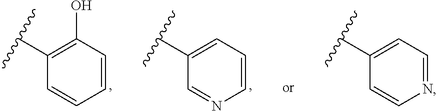
if R₁ is 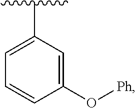
then R₂ is 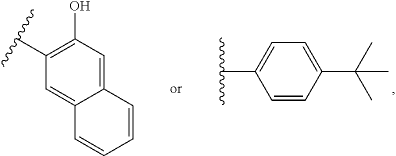
if R₁ is 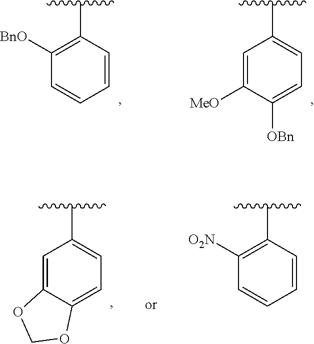
then R₂ is 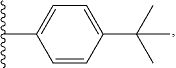
and
if R₁ is 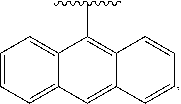
then R₂ is 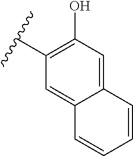
and a pharmaceutically acceptable carrier therefor.

2. The pharmaceutical composition of claim 1, wherein said inhibition occurs in respect of at least one Class B or Class Dβ-lactamase enzyme.

3. The pharmaceutical composition of claim 2, wherein the Class B β-lactamase enzyme is selected from IMP-1 and VIM-2.

4. The pharmaceutical composition of claim 2, wherein the Class D β-lactamase enzyme is selected from OXA-10 and OXA-45.

5. The pharmaceutical composition of any one of claims 2 to 4 wherein R₁ is

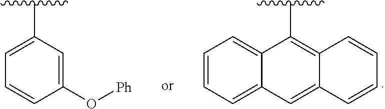

6. The pharmaceutical composition of claim 2, wherein said inhibition occurs in respect of at least one Class B enzyme, and R₁ is selected from

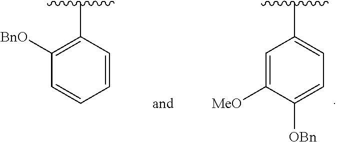

7. The pharmaceutical composition of claim 6, wherein the Class B enzyme is IMP-1.

8. The pharmaceutical composition of any one of claims 1 to 7 for use in the manufacture of a medicament for the treatment of bacterial infections.

9. The pharmaceutical composition of any one of claims 1 to 8 further comprising a pharmaceutically acceptable β-lactam antibiotic.

10. The pharmaceutical composition of claim 9, wherein the β-lactam antibiotic is selected from a penicillin, a cephalosporin, an oxacephem, a penem, or a carbapenem.

11. The pharmaceutical composition of claim 10, wherein the β-lactam antibiotic is pipericillin.

12. A method of treating a bacterial infection comprising administering to a mammalian patient in need of such treatment a compound of formula (I): 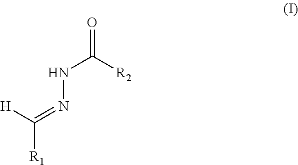
wherein
R₁ is selected from 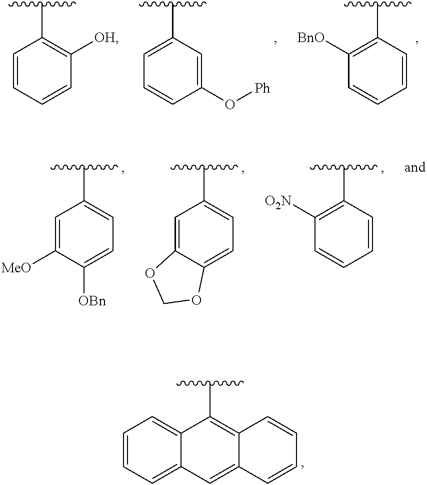
R₂ is selected from 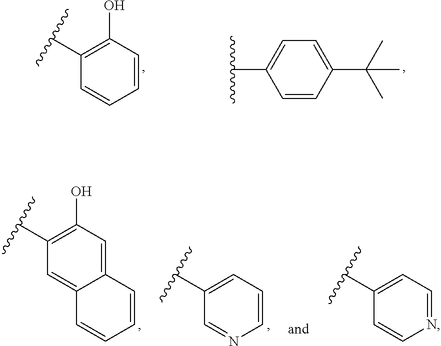
with the proviso that:
if R₁ is 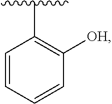
then R₂ is selected from 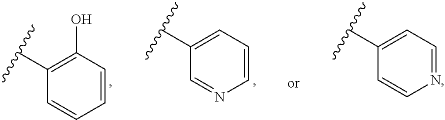
if R₁ is 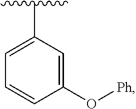
then R₂ is 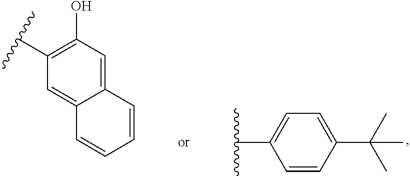
if R₁ is 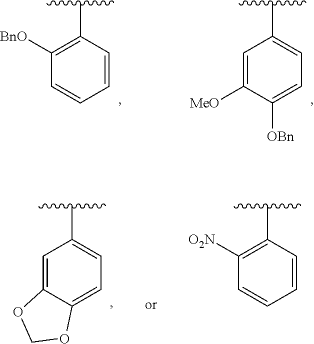
then R₂ is 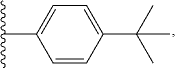
and
if R₁ is 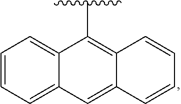
then R₂ is 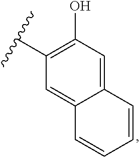
in combination with a pharmaceutically acceptable β-lactam antibiotic in an amount which is effective for treating the bacterial infection.

13. The method of claim 12, wherein said bacterial infection comprises bacteria expressing at least one Class B or Class D β-lactamase enzyme.

14. The method of claim 13, wherein the Class B β-lactamase enzyme is selected from IMP-1 and VIM-2.

15. The method of claim 13, wherein the Class D β-lactamase enzyme is selected from OXA-10 and OXA-45.

16. The method of any one of claims 13 to 15 wherein R₁ is

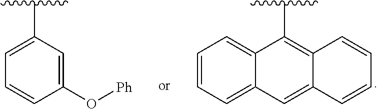

17. The method of claim 13, wherein said bacteria express at least one Class B enzyme, and R_{I} is selected from

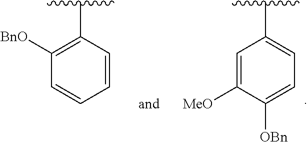

18. The method of claim 17, wherein the Class B enzyme is IMP-1.

19. The method of any one of claims 12 to 18, wherein the β-lactam antibiotic is selected from a penicillin, a cephalosporin, an oxacephem, a penem, or a carbapenem.

20. The method of claim 19 wherein the β-lactam antibiotic is pipericillin.

21. A method of inhibiting a β-lactamase enzyme, the method comprising contacting the β-lactamase enzyme with a compound of formula (I): 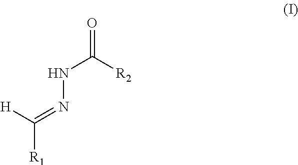
wherein
R₁ is selected from 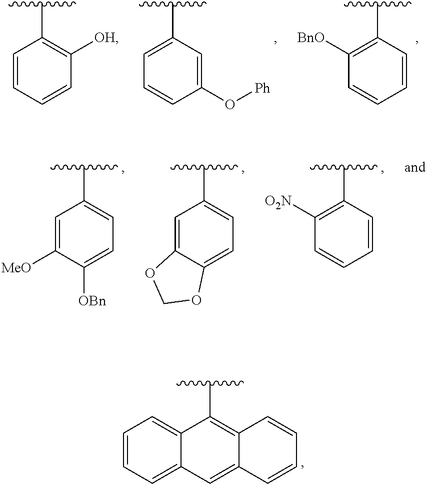
R₂ is selected from 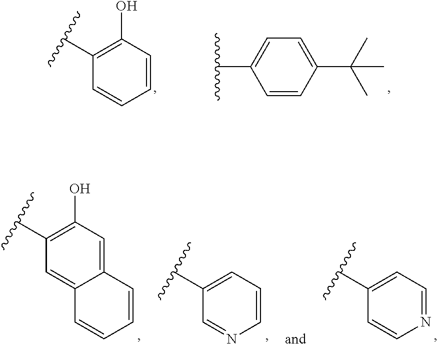
with the proviso that:
if R₁ is 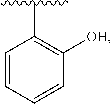
then R₂ is selected from 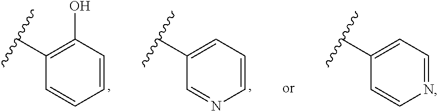
if R₁ is 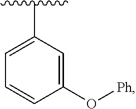
then R₂ is 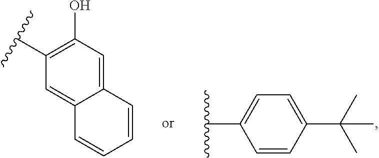
if R₁ is 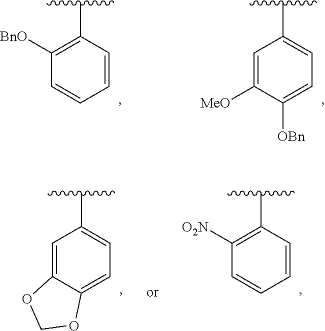
then R₂ is 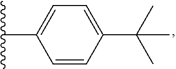
and
if R₁ is 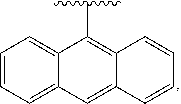
then R₂ is

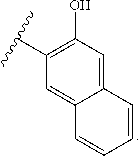

22. The method of claim 21, wherein said β-lactamase enzyme is a Class B or Class D β-lactamase enzyme.

23. The method of claim 22, wherein the Class B β-lactamase enzyme is selected from IMP-1 and VIM-2.

24. The method of claim 22, wherein the Class D β-lactamase enzyme is selected from OXA-10 and OXA-45.

25. The method of any one of claims 22 to 24 wherein R₁ is

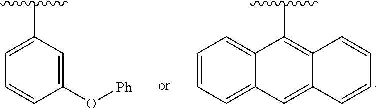

26. The method of claim 22, wherein said inhibition occurs in respect of at least one Class B β-lactamase enzyme, and R_{I} is selected from

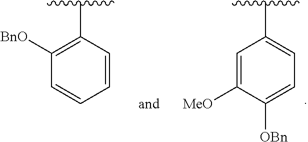

27. The method of claim 26, wherein the Class B enzyme is IMP-1.
